(19) United States
(12) Patent Application Publication
(10) Pub. No.: US 2026/0294828 A1
(43) Pub. Date: Oct. 1, 2026

(54) CALIXARENE-BASED DELIVERY SYSTEM AND METHOD OF USE

(71) Applicant: QUANTOOM BIOSCIENCES S.A., Nivelles (BE)

(72) Inventors: Aurélien VANDER STRAETEN, Nivelles (BE); Robin BEVERNAEGIE, Nivelles (BE)

(21) Appl. No.: 19/479,327

(22) PCT Filed: Apr. 29, 2024
(86) PCT No.: PCT/EP2024/061814

§ 371 Date: Oct. 27, 2025

(30) Foreign Application Priority Data
Apr. 28, 2023 (EP) 23170808.2

(51) Int. Cl. A61K 9/51; A61K 39/00; A61K 39/205; A61K 39/215; A61P 37/04

(52) U.S. Cl. CPC A61K 9/5123; A61K 39/205; A61K 39/215; A61P 37/04; A61K 2039/53; A61K 2039/55555

(57) ABSTRACT

The current invention relates to a delivery system to deliver one or more cargo to one or more cells, wherein the cargo delivery system comprises at least a calixarene, a phospholipid, an additional lipid such as sterol. The invention further relates to a method of delivering cargo to a subject using the delivery system and a pharmaceutical composition comprising the delivery system. The invention also relates to the use of a calixarene in an immunogenic composition, wherein said composition comprises an immunogenic component encapsulated in a lipid nanoparticle (LNP) comprising said calixarene and wherein said LNP has an adjuvant effect in said immunogenic composition. The invention also relates to a vaccine, wherein said vaccine comprises an immunogenic component encapsulated in a lipid nanoparticle, wherein said lipid nanoparticle comprises at least one calixarene molecule and said lipid nanoparticle acts as an adjuvant in said vaccine. The invention also relates to a method of preparing an immunogenic composition and a composition comprising a lipid nanoparticle (LNP) adjuvant comprising calixarene.

### FIELD OF THE INVENTION

The present invention relates to a delivery system, specifically a calixarene-based delivery system for transporting various cargos, such as nucleic acids, proteins, chemical substances, and polysaccharides, to target cells. The invention also encompasses a method of delivering cargo to a subject using the described delivery system.

### BACKGROUND

The efficient and targeted delivery of therapeutic agents, such as nucleic acids, proteins, and chemical substances, to cells is a critical aspect of modern medicine. Various delivery systems have been developed to enhance the efficacy and safety of these agents. However, there is a continuous need for improved delivery systems that can effectively deliver diverse types of cargo to target cells.

RNA therapeutics comprise a rapidly expanding category of drugs that will change the standard of care for many diseases and actualize personalized medicine. These drugs are cost effective, relatively simple to manufacture, and can target previously undruggable pathways. However, employing nucleic acids as therapeutics is challenging because they are susceptible to degradation by nucleases, contribute to immune activation and have unfavorable physicochemical characteristics that prevent facile transfection into cells. Safe and effective nucleic acid therapeutics therefore require sophisticated delivery platform technologies.

Lipid nanoparticles are the leading technology for nonviral nucleic acid delivery. Naked RNA is quickly degraded after administration by cellular ribonucleases (RNases). LNPs slow down the degradation process to ensure RNA stability while also promoting cellular internalization via endocytosis and allowing intracellular release of RNA into the cytoplasm for translation by cellular machinery.

The composition of LNPs typically includes an ionizable cationic lipid and three neutral helper lipids: phospholipid, cholesterol, and lipid-anchored polyethylene glycol (PEGylated lipid). The ionizable cationic lipids are complexed with polyanionic RNA through ion pairing interactions to enable its encapsulation by the neutral lipids and facilitate cellular uptake and endosomal escape.

LNPs were initially optimized for formulating siRNA (~23 nt) and have recently evolved to encapsulate larger RNA-agents, including mRNA (~1000 nt). Recently, there has been increased activity for even larger RNA payloads, such as self-amplifying RNA (saRNA). saRNA is a promising alternative to mRNA as it has been shown to induce immune responses with up to 100-fold lower doses and extended protein expression in vivo compared to mRNA. However, saRNA (~10 000 nt) is larger than mRNA (~1000 nt) and has more secondary structure, making it more difficult to encapsulate and deliver. The inherent chemical and structural differences between mRNA and saRNA in terms of length, stability, and charge density suggests that LNP delivery formulations for saRNA may require conditions significantly different from those developed for mRNA delivery.

The ionizable lipid is considered to be the most important factor for improving encapsulation efficiency, as this is the component responsible for complexing with RNA cargo. However, in order to obtain a sufficient encapsulation efficiency for saRNA, high amounts of ionizable lipids are necessary (for instance 20 times the amount of ionizable lipid compared to the amount of saRNA).

Optimizing the efficiency of cargo encapsulation will minimize waste of expensive materials (i.e. RNA), and generate a higher concentration drug formulation.

Besides influencing encapsulation efficiency, cellular uptake and promotion of endosomal escape of nucleic acid cargo, the various constituents of the LNP are also important to facilitate monodisperse nanoparticle formation and improve nanoparticle stability. Polydispersity index (PDI) is a normalized value that indicates nanoparticle size range in a sample, and is a useful indicator of sample quality. In samples with high dispersity, larger particles in the distribution will tend to aggregate and sediment, which leads to diminished effective RNA concentration and inconsistent dosing. Typically, LNP formulations developed for biological application should have a PDI below 0.2, which indicates the colloid is acceptably monodisperse. Monodispersity of nanoparticle drugs is crucial to ensure the consistent behavior of the intended drug, as size influences how particles interact with the body. However, in order to obtain a monodisperse population of LNPs, especially for those having an saRNA cargo, high amounts of ionizable lipids are necessary.

The present invention aims to resolve at least some of the problems and disadvantages mentioned above.

### SUMMARY OF THE INVENTION

The present invention provides a delivery system for delivering one or more cargos to one or more cells according to claim 1. The delivery system comprises a calixarene, a phospholipid, an additional lipid such as a sterol, and optionally a PEGylated lipid. Optionally, the delivery system may also include an ionizable lipid and/or a cationic lipid. The calixarene may be an ionizable calixarene, a cationic calixarene, or any combination thereof.

The delivery system can include a specific concentration of calixarene, phospholipid, additional lipid such as sterol, PEGylated lipid, ionizable lipid, and cationic lipid. Additionally, various types of phospholipids, sterols, PEGylated lipids, and cationic lipids can be incorporated into the delivery system, as described in the claims. The delivery system may be tailored to include ionizable or cationic calixarenes, particularly depending on the desired cargo and target cells.

The invention also provides a method of delivering one or more cargos to a subject, comprising administering aforementioned delivery system to a subject. The cargo may be selected from a group consisting of a nucleic acid, a protein, a chemical substance, a polysaccharide, and combinations thereof. Preferably, the cargo is a nucleic acid, more preferably RNA or DNA.

The invention further provides a pharmaceutical composition comprising aforementioned delivery system for use in the prevention and/or treatment of a disease or disorder in a subject.

The invention further provides a use of a calixarene in an immunogenic composition. In further aspects, the invention relates to a vaccine; a method of preparing an immunogenic preparation; a composition comprising a lipid nanoparticle (LNP) adjuvant comprising calixarene and an antigen; and a method for enhancing an immune response.

The present invention offers an improved delivery system that can effectively deliver diverse types of cargo to target cells, with potential applications in various therapeutic settings.

### DESCRIPTION OF FIGURES

The following description of the figures of specific embodiments of the invention is merely exemplary in nature and is not intended to limit the present teachings, their application or uses. Throughout the drawings, corresponding reference numerals indicate like or corresponding parts and features.

FIG. 1 shows chemical structures of ionizable calixarenes used in the delivery system according to an embodiment of the current invention.

FIG. 2 shows a visualization of the estimated model (average+95% confidence interval) of in vivo luminescence as a function of the (lipid+CX)/RNA mass ratio, the helper mass ratio and the PEG lipid mass ratio in a delivery system according to an embodiment of the current invention.

FIG. 3 shows the in vivo protein expression at 3 h, 6 h and 9 h after the intramuscular injection of 1 μg of Fluc mRNA into both left and right hindlimb of mice (geometric mean signal with 95% confidence intervals) using various delivery systems according to embodiments of the current invention.

FIG. 4 shows saRNA-FLuc encapsulation in nanoparticles according to an embodiment of the invention having a different CX/RNA mass ratio and the resulting in vitro protein expression after transfection of these nanoparticles.

FIG. 5 shows in vivo saRNA-Fluc delivery efficiency of a delivery system according to an embodiment of the current invention compared to a reference delivery system without a calixarene.

FIG. 6 shows virus neutralizing titers (VNT) in serum measured after 15, 35 and 64/65 days in mice which were vaccinated using a delivery system according to an embodiment of the invention comprising an ionizable calixarene and loaded with mRNA encoding for the G-protein of the Rabies virus. A 21 days prime-boost regimen using either 0.6 μg or 2.5 μg of mRNA per dose was used.

FIG. 7 shows a cryo-TEM image of a delivery system of the current invention encapsulating mRNA.

FIG. 8 shows chemical structures of synthesized cationic calixarenes for use in delivery systems according to an embodiment of the invention.

FIG. 9 shows a design of experiment (DoE) to model the effect of cationic calixarene mass per LNP (expressed as milligrams of cationic calixarene per milligram of RNA dose) on physicochemical properties of the resulting LNP according to an embodiment of the invention.

FIG. 10 shows virus neutralizing titers (VNT) in serum measured after 15 and 35 days in mice which were vaccinated using a delivery system according to an embodiment of the invention comprising a cationic calixarene (either CX4 or CX12), a PEGylated lipid (either DMG-PEG2000 or DSG-PEG2000), DOPE and cholesterol. The delivery system was loaded with mRNA encoding for the G-protein of the Rabies virus. A 21 days prime-boost regimen using either 0.6 μg or 2.5 μg of mRNA per dose was used.

FIG. 11 shows the intensity distribution of an LNP according to an embodiment of the invention comprising DLin-DMA and a cationic calixarene (DLin-DMA: DOPE: cholesterol:DMG-PEG2000: CX12 LNP in 20 mM TRIS buffer) encapsulating saRNA encoding for the spike protein of SARS-COV-2 (FIG. 11A) and of a reference LNP not comprising a cationic calixarene (SM-102: DSPC: cholesterol:DMG-PEG2000 (50:10:38.5:1.5 molar ratio)) encapsulating saRNA encoding for an undisclosed gene of similar length (FIG. 11B).

FIG. 12 shows the log 10 luminescence at the injection site 6 hours after intramuscular (IM) injection of LNPs where either DOTAP is present in the LNP or where a cationic calixarene is present in the LNP according to an embodiment of the current invention.

FIG. 13 shows IgG levels (points correspond to individual observations and lines correspond to the geometric mean with 95% confidence intervals) for spike protein and the receptor binding domain (RBD) of the spike protein. All animals for DODAP/DOTAP had levels below limits of quantification, in contrast to when a calixarene is present in the LNP according to an embodiment of the current invention.

FIG. 14 shows VNTs serum levels measured after 35 (after prime-boost) for the DODAP: CX12 LNP encapsulating mRNA encoding for the G-protein of the Rabies virus according to an embodiment of the invention. A 2.5 μg dose was administrated IM in BALB/c mice.

FIG. 15A shows VNTs serum levels measured after 15 (after prime), 35 and 64/65 (after boost) days for each LNP, including LNPs according to an embodiment of the current invention.

FIG. 15B shows the virus neutralizing titers (VTNs) in serum measured after 15 (after prime), 35 and 65 days (after boost) days in mice which were vaccinated using SM-102 LNPs (control), DLin-DMA LNPs and DLin-DMA/CX4 LNPs, the latter being a delivery system according to an embodiment of the current invention. A 21 days prime-boost regimen using 2.5 μg of mRNA per dose was used (RNA encoding for the glycoprotein G from the Rabies virus. The results are shown as a comparison with the control delivery system (SM-102 LNPs). UDL=upper decision limit, LDL=lower decision limit. All points crossing the limits indicate a significant difference as compared to the control.

FIG. 16 shows the chemical structure of a cationizable calixarene (CX5) which can be used in LNPs according to an embodiment of the current invention.

FIG. 17 shows VNTs serum levels measured after 15 (after prime), 35 and 65 (after boost) days for each nanoparticle (A: 0.6 μg doses, B: 2.5 μg doses IM administration in BALB/c), including an LNP where a cationizable calixarene was used according to an embodiment of the current invention.

### DETAILED DESCRIPTION OF THE INVENTION

The following detailed description provides further information about the calixarene-based delivery system and the method of use according to the present invention. The invention encompasses a delivery system for delivering various types of cargo to target cells and a method for administering the delivery system to a subject.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt”, % (w/w) or “wt %”, here and throughout the description unless otherwise defined, refers to the relative weight of the respective component based on the overall weight of the formulation (for instance the overall weight of the delivery system excluding the cargo (e.g. RNA) and excluding other excipients (e.g. sucrose, TRIS, etc.)). The components included in “the formulation” and making up “the overall weight of the formulation” are dependent on the context. In the current invention, when referring to the mass fraction of the calixarene component or the lipid component relative to the overall weight of the formulation, the weight of the calixarene component or the lipid component is relative to the weight of the total sum of the calixarenes and lipids present in the formulation, more specifically, present in the delivery system, more specifically present in the lipid nanoparticle. As such, in said instance, “the overall weight of the formulation”, does not include the cargo (e.g. RNA), nor other excipients (e.g. sucrose, TRIS, etc.)).

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

As used herein, the term “optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “lipid” refers to a group of organic compounds that comprise, but are not limited to, esters of branched or unbranched fatty acids and are generally characterized by being poorly soluble in water, but soluble in many organic solvents. Lipids are usually divided into at least three classes: (1) “simple lipids,” which include fats and oils as well as waxes; (2) “compound lipids,” which include phospholipids or glycolipids; and (3) “derived lipids” such as steroids.

In the context of the present invention, the term “sterol”, also known as steroid alcohol, is a subgroup of steroids that occur naturally in plants, animals, and fungi, or can be produced by some bacteria.

The term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

In the context of the present disclosure the term “ionizable” in the context of a compound or lipid means the presence of any uncharged group in said compound or lipid which is capable of associating with an ion (usually an H⁺ ion) and thus itself becoming positively charged (also referred to as “cationizable”). Alternatively, any uncharged group in said compound or lipid may yield an ion (usually an H⁺ ion) and thus becoming negatively charged. In the context of the present disclosure any type of ionizable lipid can suitably be used.

The term “lipid nanoparticle” refers to a particle having at least one dimension in the order of nanometers (e.g., 1-1,000 nm) and comprises a plurality of lipid molecules physically associated with each other by intermolecular forces. An active agent or therapeutic agent, such as a nucleic acid, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

The term “oligonucleotide” or “polynucleotide” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of self-amplifying RNA (saRNA), small hairpin RNA (shRNA), messenger RNA (mRNA), antisense RNA, miRNA, micRNA, multivalent RNA, dicer substrate RNA or viral RNA (vRNA), guide RNA (gRNA), and combinations thereof.

Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof.

As used herein, the term N/P ratio, or N: P ratio is the molar ratio of nitrogen atoms in a complexing lipid (or other chemical structure comprising at least one amino group, used for encapsulation) to phosphate groups in an RNA. This ratio describes the interaction between the cationic charge of the amino (N+) group in amino-lipid (or other chemical structure) to the anionic charge of the phosphate (PO4−) groups in the backbone of nucleic acids and is the basis of the complexation of RNA with the amino-lipid (or other chemical structure comprising at least one amino group). The N/P ratio of a lipid/nucleic acid complex can potentially influence other properties such as its net surface charge, size, and stability.

“Effective amount” or “therapeutically effective amount” refers to that amount of a pharmaceutical composition, which, when administered to an animal, preferably a mammal, more preferably a human, is sufficient to effect treatment in the mammal, preferably a human. The amount of the pharmaceutical composition of the invention which constitutes a “therapeutically effective amount” will vary depending on the compound, the condition and its severity, the manner of administration, and the age of the animal to be treated, but can be determined routinely by one of ordinary skill in the art having regard to his own knowledge and to this disclosure.

The term “self-replicating and “self-amplifying” as used herein are used interchangeably and relate to molecules such as RNA comprising within their sequence specific signals or signature sequences that allow the self-replication or self-amplification of said molecule.

“Encapsulation efficiency” as used herein refers to the ratio of encapsulated RNA to total RNA in the sample. While there are a handful of methods to characterize encapsulation efficiency of an LNP formulation for its cargo, modified RiboGreen assay is by far the most common. RiboGreen is a dye developed for quantitation of RNA in which fluorescence is produced upon nucleic acid binding. For determination of encapsulation efficiency in LNPs, RiboGreen is first added to the sample with LNPs intact to measure the unencapsulated RNA concentration. A detergent solution (e.g. Triton X-100) is then added to disrupt the nanoparticles, releasing encapsulated RNA, and the total amount of RNA in the sample is calculated from RiboGreen fluorescence. Encapsulated RNA is calculated by subtracting unencapsulated RNA from total RNA and encapsulation efficiency is then taken as the ratio of encapsulated RNA to total RNA in the sample.

“Adjuvants” are usually defined as compounds that can increase and/or modulate the intrinsic immunogenicity of an antigen. To reduce negative side effects, new vaccines have a more defined composition that often leads to lower immunogenicity compared with previous whole-cell or virus-based vaccines. Adjuvants are therefore required to assist new vaccines to induce potent and persistent immune responses, with the additional benefit that less antigen and fewer injections are needed.

“Adjuvant/adjuvant component”: An adjuvant or an adjuvant component in the broadest sense is typically a (e.g. pharmacological or immunological) agent or composition that may modify, e.g. enhance, the efficacy of other agents, such as a drug or vaccine. Conventionally the term refers in the context of the invention to a compound or composition that serves as a carrier or auxiliary substance for immunogens and/or other pharmaceutically active compounds. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immuno potentiators, antigenic delivery systems or even combinations thereof. The term “adjuvant” is typically understood not to comprise agents which confer immunity by themselves. An adjuvant assists the immune system unspecifically to enhance the antigen-specific immune response by e.g. promoting presentation of an antigen to the immune system or induction of an unspecific innate immune response. Furthermore, an adjuvant may preferably e.g.

modulate the antigen-specific immune response by e.g. shifting the dominating Th2-based antigen specific response to a more Th1-based antigen specific response or vice versa and/or by inducing of mucosal immune responses and/or increased IgA titers. Accordingly, an adjuvant may favourably modulate cytokine expression/secretion, antigen presentation, type of immune response etc.

Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccinees. These may be co-administered by any route, e.g., intramuscular/, subcutaneous, IV or intradermal injections.

The term “antigen” as used herein refers to a substance which may be recognized by the immune system and may be capable of triggering an antigen-specific immune response, e.g. by formation of antibodies or antigen-specific T-cells as part of an adaptive immune response.

“Epitope” (also called “antigen determinant”) as used herein refers to T cell epitopes that may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule. B cell epitopes are typically fragments located on the outer surface of (native) protein or peptide antigens.

“A vaccine” as used herein refers to a prophylactic or therapeutic material providing at least one antigen or antigenic function. The antigen or antigenic function may stimulate the body's adaptive immune system to provide an adaptive immune response.

“Immunogenicity” is the ability of a foreign substance, such as an antigen, to provoke an immune response in the body of a human or other animal. An “immunogenic composition” as used herein is able to provoke an immune response in the body of a human or other animal.

### Compositions, Pharmaceutical Compositions And Formulations

The current invention is directed to a (pharmaceutical) composition that serves as a delivery system to deliver one or more cargo to one or more cells, wherein the delivery system comprises at least a calixarene, a phospholipid, an additional lipid such as a sterol, and optionally a PEGylated lipid.

In a preferred embodiment, the delivery system is a lipid nanoparticle (LNP) comprising at least a calixarene, a phospholipid, a sterol, and a PEGylated lipid.

Lipid nanoparticles are the leading technology for nonviral nucleic acid delivery. Naked RNA is quickly degraded after administration by cellular ribonucleases (RNases). LNPs slow down the degradation process to ensure RNA stability while also promoting cellular internalization via endocytosis and allowing intracellular release of RNA into the cytoplasm for translation by cellular machinery.

The composition of LNPs known from the art typically includes an ionizable cationic lipid and three neutral helper lipids: phospholipid, cholesterol, and lipid-anchored polyethylene glycol (PEGylated lipid). The ionizable cationic lipids are complexed with polyanionic RNA through ion pairing interactions to enable its encapsulation by the neutral lipids and facilitate cellular uptake and endosomal escape.

Cationic or ionizable lipids (under acidic pH) promote ion pairing interaction with the negatively charged backbone of nucleic acids. This ion pairing interaction facilitates encapsulation of nucleic acid cargo within the electron dense LNP core. Permanently cationic lipids can cause unwanted toxicity and immune response issues, resulting in the increasing adoption of ionizable lipids. Ionizable lipids are positively charged during LNP formation, while mostly neutral at physiological pH. Maintaining neutral pH during circulation helps prevent adsorption of negatively charged biological molecules, thereby preventing rapid clearance by immune cells and increasing circulation time. The ionizable lipid also facilitates nucleic acid cargo release due to electrostatic interactions with the anionic endosomal membrane, which occurs because of the protonated state of the ionizable lipid under acidic pH within the acidic microenvironment of the endosome.

LNPs were initially optimized for formulating siRNA (~23 nt) and have recently evolved to encapsulate larger RNA-agents, including mRNA (~1000 nt). Recently, there has been increased activity for even larger RNA payloads, such as self-amplifying mRNA (saRNA). saRNA is a promising alternative to mRNA as it has been shown to induce immune responses with up to 100-fold lower doses and extended protein expression in vivo compared to mRNA. However, saRNA (~10 000 nt) is larger than mRNA (~1000 nt) and has more secondary structure, making it more difficult to encapsulate and deliver. The inherent chemical and structural differences between mRNA and saRNA in terms of length, stability, and charge density suggests that LNP delivery formulations for saRNA may require conditions significantly different from those developed for mRNA delivery.

The ionizable lipid is considered to be the most important factor for improving encapsulation efficiency, as this is the component responsible for complexing with RNA cargo. However, in order to obtain a sufficient encapsulation efficiency for saRNA, high amounts of ionizable lipids is necessary (for instance 20 times the amount of ionizable lipid compared to the amount of saRNA).

Besides influencing encapsulation efficiency, cellular uptake and promotion of endosomal escape of nucleic acid cargo, the various constituents of the LNP are also important to facilitate monodisperse nanoparticle formation and improve nanoparticle stability. Polydispersity index (PDI) is a normalized value that indicates nanoparticle size range in a sample, and is a useful indicator of sample quality. In samples with high dispersity, larger particles in the distribution will tend to aggregate and sediment, which leads to diminished effective RNA concentration and inconsistent dosing. Typically, LNP formulations developed for biological application should have a PDI below 0.2, which indicates the colloid is acceptably monodisperse. Monodispersity of nanoparticle drugs is crucial to ensure the consistent behavior of the intended drug, as size influences how particles interact with the body.

However, in order to obtain a monodisperse population of LNPs having an saRNA cargo, high amounts of ionizable lipids are necessary.

The current invention is centered around delivery systems (LNPs, more specifically) comprising at least a calixarene.

Calixarenes are macrocyclic molecules that are known for their ability to encapsulate and transport various cargos, such as nucleic acids, proteins, and chemical substances. Calixarenes are macrocyclic molecules based on methylene-linked phenols. In the present invention, the delivery system includes a calixarene, which may be an ionizable calixarene, a cationic calixarene, or any combination thereof.

This modification is expected to change completely the behavior of the resulting delivery systems, especially their encapsulation and releasing capabilities.

In the current invention, the size of the calixarene can vary depending on the plurality of desired features, however, the calixarene is preferably a calix[4] arene.

Calix[4] arenes display several key features that makes them suitable for nucleic acid delivery. The most important is their natural cone-shaped conformation that was found crucial for lipid nanoparticles/ionizable lipids to achieve high endosomal escape and favor the release of RNA in the cytosol.

As described above, a permanently charged cationic component can cause unwanted toxicity and immune response issues, which resulted in the incorporation of ionizable lipids in the traditional LNPs known from the state of the art.

In an embodiment, the current invention now provides a delivery system wherein an ionizable calixarene is used in RNA lipid nanoparticles (RNA-LNPs) to replace the traditional ionizable lipid. The resulting delivery system hence comprises 4 components: an ionizable calixarene, a helper lipid, a sterol, and a PEG lipid.

Calixarenes are platforms which facilitate the synthesis of ionizable compounds with multiple amine heads, meaning that the charge density (number of amines/molecule) could be increased easily. Besides reducing the amount of ionizable component necessary for efficiently encapsulating nucleic acids, this property also facilitates the encapsulation of very long RNA, such as self-amplifying RNA (saRNA), by increasing the number of amines without changing the mass ratio between the ionizable component and RNA, while this task remains challenging with the current LNP technology having a formulation that does not comprise calixarenes.

In an alternative embodiment, the current invention provides a delivery system wherein a cationic calixarene is incorporated as a fifth component into lipid nanoparticles (LNPs) made of an ionizable lipid, a helper lipid, a sterol, and a PEG lipid. As described above and similar to the ionizable calixarenes, the charge density (number of amines/molecule) could be increased easily in cationic calixarenes, facilitating the encapsulation of nucleic acids, especially that of very long RNA, such as self-amplifying RNA (saRNA). Furthermore, by incorporating a cationic calixarene into a traditional LNP (“5 component delivery system”) the current invention also allows (i) to minimize the inherent toxicity of these cationic components by combining them with non-toxic biocompatible lipids, and (ii) to reduce the non-specific adsorption of proteins that usually limits their efficacy.

The calixarene concentration in the delivery system can range from 0.1-60 mol %, depending on the desired characteristics of the delivery system. Such as, the calixarene concentration in the delivery system can range from 0.1-50 mol %, from 0.1-44% mol %, from 0.1-40 mol %, from 0.1-30 mol %, from 0.1-20 mol %, from 0.1-10 mol %, from 0.1-5 mol % or from 1-60 mol %, from 10-60 mol %, from 20-60 mol %, from 30-60 mol %, from 35-60 mol % or any ranges and subranges therein between.

In the current invention, the size of the calixarene can vary depending on the plurality of desired features, such as, the precise conformation of the calixarene structure e.g. cone-shape or other conformations, cargo of choice, and/or target cells. According to disclosure, not limiting but preferred calixarene structures includes calixarenes comprising 4 monomers, 6 monomers or 8 monomers, namely calix[4] arenes, calix[6] arenes, calix[8] arenes. In preferred embodiments, calixarene is calix[4] arenes.

In a preferred embodiment, the delivery system according to the current invention comprises a calix[4] arene.

In an embodiment, a calix[4] arene as used in the delivery system of the invention can be a compound of Formula (I), wherein “A” represents a hydrophobic part of the molecule and “B” represent a head group (either ionizable or cationic) which forms the hydrophilic part of the molecule.

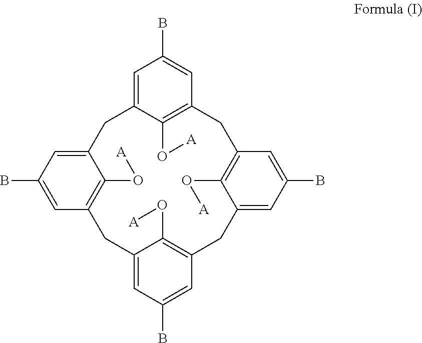

In an embodiment, the cationic or ionizable heads can be linked to the macrocycle through various biodegradable and non-biodegradable groups.

In an embodiment and referring to Formula (I), the calixarene used in the current invention comprises 4 identical B-groups. In another embodiment, the calixarene used in the current invention comprises 3 identical B-groups and one B-group which is different. In another embodiment, the calixarene used in the current invention comprises 2 identical B-groups and 2 other identical B-groups. The same holds true for the A-groups. As can be understood, many configurations are possible.

In embodiments, if a cationic calixarene is used, the concentration can be between 0.1-10 mol % of said delivery system. For example, the concentration of cationic calixarene can be between 0.1-10 mol %, 0.5-9 mol %, 1-8 mol %, 2-7 mol %, 3-6 mol %, 4-5 mol % or 0.1-9 mol %, 0.1-8 mol %, 0.1-7 mol %, 0.1-6 mol %, 0.1-5 mol %, 0.1-4 mol %, 0.1-3 mol %, 0.1-2 mol %, 0.1-1 mol %, or 0.5-10 mol %, 1-10 mol %, 2-10 mol %, 3-10 mol %, 4-10 mol %, 5-10 mol %, 6-10 mol %, 7-10 mol %, 8-10 mol %, 9-10 mol % and all ranges and subranges therein between. In embodiments, the number of cationic sites (heads) of the calixarene is ranging between 1 to 8, e.g., calixarene can have 8, 7, 6, 5, 4, 3, 2, or 1 cationic sites, preferably 1 to 4 cationic sites.

In embodiments, the concentration of an ionizable calixarene is 10-40 mol % of said delivery system.

In embodiments, the concentration of an ionizable calixarene is 10-60 mol % of said delivery system.

For example, the concentration of ionizable calixarene can be between is 10-60 mol %, 10-50 mol %, 10-45 mol %, 10-40 mol %, 10-35 mol %, 10-30 mol %, 10-25 mol %, 10-20 mol %, 10-15 mol % or 15-60 mol %, 20-60 mol %, 25-60 mol %, 30-60 mol %, 35-60 mol % or 15-55 mol %, 15-45 mol %, 15-40 mol %, 20-35 mol %, 20-30 mol %, 20-25 mol %, 25-30 mol %, or any ranges and subranges therein between.

Phospholipids are essential components of biological membranes and can be used in the delivery system to enhance stability and biocompatibility. Suitable phospholipids for the delivery system can be selected from the group of phosphatidylcholines, phosphatidylethanolamines, and sphingolipids. The concentration of phospholipids in the delivery system can range from 1 to 45 mol %. For example, the phospholipid concentration in the delivery system can range from 1-40 mol %, from 1-30 mol %, from 1-20 mol %, from 1-10 mol %, from 1-5 mol %, or from 5-45 mol %, from 10-45 mol %, from 20-45 mol %, from 30-45 mol %, from 35-45 mol %, or from 5 to 40 mol %, from 10 to 35 mol %, from 15 to 30 mol %, from 20 to 30 mol % and any ranges and subranges therein between.

In a preferred embodiment, the additional lipid confers rigidness to the delivery system. In an embodiment, the additional lipid can be a sterol, a fatty acid, a glycerol monooleate or a trioleate or any short molecule saturated (chosen for their rigidity property). In embodiments, the short saturated molecule is less than 17 carbon (C) long e.g., at most 16 C, at most 15 C, at most 14 C, at most 13 C, at most 12 C, at most 11 C, at most 10 C, at most 9 C, at most 8 C, at most 7 C, at most 6 C, at most 5 C, at most 4 C, at most 3 C atoms long, preferably between 10 C and 3 C atoms long.

In an embodiment, the additional lipid (for instance a sterol compound) is present in the composition according to the current invention in a concentration of 20-70 mol %. In embodiments, the additional lipid concentration can range between 20-60 mol %, 20-50 mol %, 20-40 mol %, 20-30 mol %, 20-25 mol % or 30-70 mol %, 40-70 mol %, 50-70 mol %, 60-7-mol %, 65-70 mol %, and all the ranges and subranges therein between, preferably 35-60 mol %, preferably 40-60 mol %, more preferably 42-57 mol %.

In preferred embodiments, the additional lipid is a sterol. Sterols are known to modulate membrane fluidity and stability, making them important constituents of the delivery system. Suitable sterols for the delivery system according to disclosure can be selected from the group of cholesterol, sitosterol, sitosterol-amino acid conjugates, stigmastanol, campesterol, fucosterol, brassicasterol, ergosterol, 9, 11-dehydroergosterol, and hydroxycholesterol.

In embodiments, delivery system comprises a calixarene wherein said calixarene is ionizable or cationic calixarene, a phospholipid, and an additional lipid wherein the additional lipid is selected from a sterol, a fatty acid, a glycerol monooleate, a trioleate or a short saturated molecule, characterized in that, the delivery system further comprises a ionizable lipid when said calixarene is cationic.

In embodiments, the delivery system further comprises PEGylated lipid, PEG lipid or PEG conjugate to increase the circulation time of said delivery system and reduce unwanted host response.

PEGylated lipids are lipids that have been modified by the attachment of polyethylene glycol (PEG) chains, which can improve the stability and circulation time of the delivery system. Suitable PEGylated lipids for the delivery system can be selected from the group consisting of PEG-diacylglycerols (PEG-DAG), PEG-dialkyloxypropyls (PEG-DAA), PEG-phospholipids, and PEG-ceramides. PEG-ceramides are preferably chosen from a PEG-ceramides having alkyl chain with C16 and PEG with an MW from 500 to 2000 e.g., C16 PEG500, C16 PEG750, C16 PEG 1000, C16 PEG 1250, C16 PEG 1500, C16 PEG 1750, C16 PEG2000 and any ranges and subranges there in between. The concentration of PEGylated lipids in the delivery system can be adjusted as needed.

PEG is often used for its stealth functions in nanoparticle formulations because it is a hydrophilic and flexible polymer. The conjugation of PEG to the delivery system reduces the interaction of the delivery system with plasma proteins. As a result, this prevents plasma proteins from adsorbing to the surface of liposomes and uptake of delivery systems by the reticuloendothelial system (RES). The conjugation of PEG or PEGylation allows delivery systems to circulate within the body for a longer period of time, extending their circulation half-life and, consequently, increasing the accumulation of delivery systems within the target tissues, cells such as tumors and tumor cells.

In an embodiment, the delivery system does not comprise a PEG lipid, PEGylated lipid, or PEG conjugate. In an embodiment, the delivery system comprises a polypeptide such as polysarcosine instead of a PEG lipid or PEG conjugate. Polysarcosine (pSar) is a polypeptoid based on the endogenous amino acid sarcosine (N-methylated glycine). In specific embodiment, a combination of PEG lipid PEGylated lipid, or PEG conjugate and polysarcosine is used.

In embodiments, ionizable and/or cationic lipids can be included in the delivery system to enhance the interaction with negatively charged cargo molecules, such as nucleic acids, and facilitate their encapsulation and delivery.

Suitable cationic lipids can be selected from the group consisting of DOTAP (1,2-dioleoyl-3-trimethylammonium-propane), DC-cholesterol (3β-[N-(N′,N′-dimethylaminoethane)-carbamoyl]cholesterol), DORI (N-(2-hydroxyethyl)-N,N-dimethyl-2,3-bis(oleoyloxy) propan-1-aminiumbromide), DOSPA (2,3-dioleyloxy-N-(2-(sperminecarboxamido)ethyl)-N, N-dimethyl-1-propanaminium Trifluoroacetate), DOTMA ICE (imidazole cholesterol ester), (1,2-di-O-octadecenyl-3-trimethylammonium propane), or any combination thereof.

However, in a preferred embodiment, not a cationic lipid, but an ionizable lipid can be included in the delivery system.

In an embodiment wherein an ionizable lipid is used, the combined concentration of ionizable lipid and cationic calixarene is 10-60 mol % in said delivery system. In embodiments, the combined concentration of ionizable lipid and cationic calixarene is 10-60 mol %, 10-55 mol %, 10-50 mol %, 10-45 mol %, 10-40 mol %, 10-35 mol %, 10-30 mol %, 10-25 mol % 10-20 mol %, 10-15 mol % or 15-60 mol %, 20-60 mol %, 25-60 mol %, 30-60 mol %, 35-60 mol % or 15-55 mol %, 15-45 mol %, 15-40 mol %, 20-35 mol %, 20-30 mol %, 20-25 mol %, 25-30 mol %, or any ranges and subranges therein between.

Depending on the specific application and desired characteristics, the delivery system can be tailored to include specific compositions of calixarene, phospholipid, additional lipid (preferably sterol), PEGylated lipid, ionizable lipid, and cationic lipid.

In other embodiments, depending on the specific application and desired characteristics, the delivery system can be tailored to include specific compositions of or calixarene, phospholipid, additional lipid (preferably sterol), ionizable lipid, and cationic lipid. The individual components and their concentration can be as described above.

In another embodiment, the delivery system can be tailored to include specific compositions of calixarene, phospholipid, additional lipid (preferably sterol) and a PEGylated lipid. The individual components and their concentration can be as described above.

In another embodiment, the delivery system can be tailored to include specific compositions of calixarene, phospholipid, additional lipid (preferably sterol), cationic lipid, and optionally a PEGylated lipid. The individual components and their concentration can be as described above.

In another embodiment, the delivery system can be tailored to include specific compositions of one or more calixarenes, phospholipid, additional lipid (preferably sterol), ionizable lipid, and optionally a PEGylated lipid.

In an embodiment, the delivery system comprises at least an ionizable calixarene, a phospholipid, a sterol and optionally a PEGylated lipid.

In an embodiment, the delivery system comprises an ionizable calixarene, DOPE, cholesterol and PEGylated lipid.

In an embodiment, said delivery system comprises at least a cationic calixarene, a phospholipid lipid, a sterol, an ionizable lipid, and optionally a PEGylated lipid.

In an embodiment, said delivery system comprises cationic calixarene, DODAP, DOPE, cholesterol and PEGylated lipid.

In a preferred embodiment, the delivery system of the current invention comprises a nucleic acid cargo, such as mRNA or saRNA. In an embodiment, the mass of the calixarene is at most 25 times higher than the mass of the nucleic acid cargo.

### 4-Component System

As described above, in an embodiment, the current invention provides a delivery system wherein an ionizable calixarene is used in RNA lipid nanoparticles (RNA-LNPs) to replace the traditional ionizable lipid (4 components: ionizable calixarene, a helper lipid, a sterol, and a PEG lipid).

It is worth noting that the PEGylated lipid is required to obtain stable monodisperse nanoparticles that do not aggregate as the PEGylated lipid ensures the shielding of particles and stabilizes their lipid-water interface. As a result, the PEGylate lipid mass fraction must be precisely controlled; a too low amount in PEG induces the aggregation of the particles, while a too high amount of PEGylated lipid limits their transfection capabilities (see in vivo protein expression discussed in example 2 below). The presence of PEG is crucial for improved physicochemical properties of LNPs; however, the level has to be minimized as there have been recorded cases of anaphylactic shock due to PEG-induced hypersensitivity reactions (HRs); in fact, PEG is considered one of the possible causes of anaphylaxis associated with COVID-19 vaccines like the Pfizer-BioNTech and Moderna mRNA vaccines.

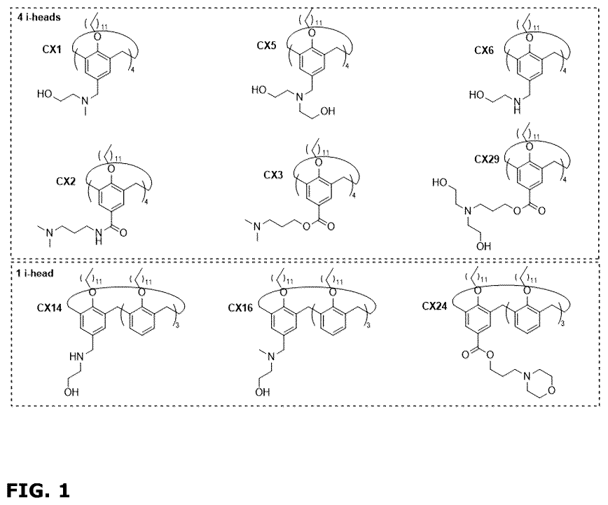

A library of ionizable calixarenes was synthetized to better understand the structure-activity relationship. Calixarenes bearing one (CX14, CX16, CX24) or four (CX1, CX2, CX3, CX5, CX6, CX29) ionizable head groups (i-head) were synthesized (FIG. 1). Ionizable heads were selected from the group of secondary amines (CX6, CX14) and ternary amines (CX1, CX2, CX3, CX5, CX16, CX24, CX29), cyclic or substituted with methyl or hydroxyethyl groups. Two biodegradable groups were also explored to link the ionizable head to the macrocyclic core and facilitate the metabolic degradation of the resulting compounds and avoid bioaccumulation (amide and ester links in CX2 and CX3/CX29 respectively). All these calixarenes were self-assembled into nanoparticles with a helper lipid (phospholipid), a sterol and a PEGylated lipid. Several helper (DOPE, DSPC) and PEG lipids (DMG-PEG2000, DSG-PEG2000) were explored. Examples of nanoparticles made with ionizable calixarenes and encapsulating a Fluc-mRNA (if not precised) or another RNA are presented in Table 1 of example 2.

Ratios between these components were defined to produce stable monodisperse nanoparticles. Interestingly, it was discovered that the N/P and molar ratios that are usually used in the field to define the resulting delivery system are not convenient for this kind of system. This is especially true when moving from 1-headed to 4-headed calixarenes, because their higher charge density (number of amines/molecule) completely changes the mass ratios between components and these parameters are of crucial importance for the stability of particles.

The inventors have observed that calix[4] arenes bearing 4 ionizable head groups (for instance depicted as the B-group in Formula I) show especially favorable characteristics for the incorporation of nucleic acids compared to calix[4] arenes bearing only 1 ionizable head group (see examples described below).

Hence, in a preferred embodiment, the delivery system according to the current invention comprises a calix[4] arene bearing 4 ionizable head groups.

Said ionizable head groups could for instance comprise secondary amines (see for instance CX6 or CX14 as depicted in FIG. 1) or ternary amines (see for instance CX1, CX2, CX3, CX5, CX16, CX24, CX29 as depicted in FIG. 1). The inventors performed in vitro potency assays in cells using LNPs comprising various ionizable calixarenes and discovered that calixarene compounds with ternary amines are more potent than calixarene compounds with secondary amines (see results in Table 2 of Example 2). In a preferred embodiment, one or more of said ionizable head groups of said calixarenes hence comprise one or more ternary amines.

As described above, said ionizable head groups of the calixarene could be different or the same. In an embodiment, said calixarene comprises 4 identical ionizable head groups.

The inventors discovered that a minimum mass ratio of [Calixarenes+Lipids]/RNA is needed to obtain stable and monodisperse particles of LNPs and optimize the in vivo protein expression of an RNA encapsulated in the LNP after administration (see Example 2). This minimum ratio RNA is ca. 20 for the 1-headed calixarenes tested, while it may be decreased to 10 with the 4-headed calixarenes tested. This interesting result shows that less material is needed to achieve similar encapsulation efficiency and monodispersity as compared to commonly used ionizable lipids (e.g., SM-102). The difference may be due to the increased charge density on the 4-headed ionizable calixarenes. In vitro potency assays of LNPs comprising ionizable calixarens in Jurkat cells using Fluc mRNA as a reporter gene were also performed. The results (see Table 2 of Example 2) provided further evidence of the superiority of the 4-headed calixarenes over the 1-headed calixarene.

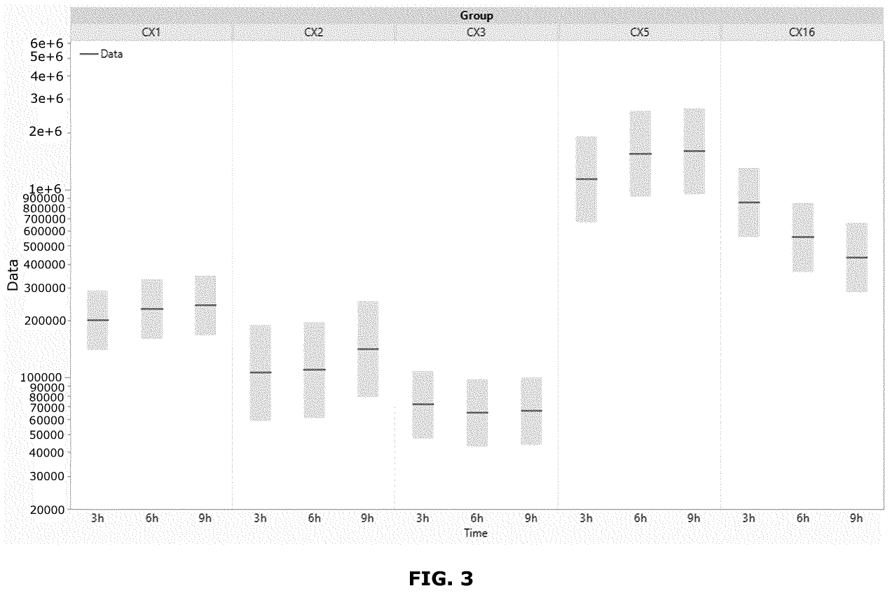

Likewise, experiments were also performed investigating the in vivo protein expression at 3 h, 6 h and 9 h after the intramuscular injection of 1 μg of Fluc mRNA encapsulated in LNPs comprising either 1-headed or 4-headed calixarenes (see Example 2, FIG. 3). Overall, this experiment indicated that the kinetic of protein expression is different between 1-headed and 4-headed calixarenes. While the signal of CX16 (1-headed) rapidly decreased, the protein expression induced by the 4-headed calixarenes (CX1, CX2, CX3 and CX5) remained stable over the different timepoints (from 3 to 9 h after injection). This result shows again the superiority of the 4-headed calixarenes over the 1-headed for achieving a stable and high protein expression. This observation is strengthened by the lower amount of 4-headed calixarenes needed to reach this performance (ca. 5 μg 4-headed CX/μg RNA vs. ca. 10 μg 1-headed CX/μgRNA).

[Calixarenes+Lipids] refers to the sum of the masses of the calixarene component and the lipid components in the LNP. In an embodiment, this mass equals the sum of the masses of the calixarene component, the helper lipid component, the sterol component and the PEGylated lipid component.

In an embodiment, when using a calixarene bearing one ionizable head group (for instance CX14, CX16, CX24 as depicted in FIG. 1) this mass ratio is below 50, more preferably below 40, more preferably below 30, more preferably below 29, 28, 27, 26, 25, 24, 23, 22, 21, such as 20. In this case, the total mass of the calixarene component plus the lipid component can be as low as only 20 times the mass of the RNA cargo.

In an embodiment, when using a calixarene bearing 4 ionizable head groups (for instance CX1, CX2, CX3, CX5, CX6, CX29 as depicted in FIG. 1) this mass ratio is below 50, more preferably below 40, more preferably below 30, more preferably 20, more preferable below 19, 18, 17, 16, 15, 14, 13, 12, 11 such as 10. In this case, the total mass of the calixarene component plus the lipid component can be as low as only 10 times the mass of the RNA cargo.

This interesting result shows that less material is needed to achieve similar encapsulation efficiency and monodispersity as compared to commonly used ionizable lipids (e.g., SM-102). The difference may be due to the increased charge density on the 4-headed ionizable calixarenes.

With respect to the helper lipid, the sterol and the PEGylated lipid, their mass fractions are conserved when moving from 1-headed to 4-headed systems. These lipid mass fractions (expressed in percent, wherein the total mass of the delivery system without cargo and other excipients represents 100%) are preferably:
Ionizable calixarene: from 10 to 60%
Helper lipid: from 5 to 35%
Sterol: from 15 to 50%
PEGylated lipid: from 4 to 24%

As such, in an embodiment, when an ionizable calixarene is used in RNA lipid nanoparticles (RNA-LNPs) to replace the traditional ionizable lipid (4 components:

ionizable calixarene, a helper lipid, a sterol, and a PEG lipid), the mass of said ionizable calixarene is 10-60% of the total mass of the delivery system.

As described above, when referring to the mass fraction of the calixarene component or the lipid component relative to the overall weight of the formulation, the weight of the calixarene component or the lipid component is relative to the weight of the total sum of the calixarenes and lipids (hence excluding the cargo (e.g. RNA) and other excipients (e.g. sucrose, TRIS, etc.)) present in the delivery system. Hence, the mass fraction refers to the mass of the lipid or calixarene with respect to the total mass of “lipid plus calixarene”. Mass is a measure of the amount of matter that an object contains.

In an embodiment of the 4 component-system, said ionizable calixarene is present in said delivery system at a mass fraction of 10-60% (w/w), such as 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or 60% (w/w) or any value in between. In an embodiment, said ionizable calixarene is present in delivery system at a mass fraction between 10-15%, 15-20%, 20-25%, 25-30%, 30-35%, 35-40%, 40-45%, 45-50%, 50-55% or 55-60% (w/w). In an embodiment, said ionizable calixarene is present in said delivery system at a mass fraction between 10-20%, 20-30%, 30-40%, 40-50% or 50-60% (w/w).

In an embodiment, when an ionizable calixarene is used in RNA lipid nanoparticles (RNA-LNPs) to replace the traditional ionizable lipid (4 components: ionizable calixarene, a helper lipid, a sterol, and a PEG lipid), the mass of said phospholipid is 5-35%, more preferably 10-30% of the total mass of the delivery system.

In an embodiment of the 4-component system, said phospholipid is present in said delivery system at a mass fraction of 5-35% (w/w), more preferably 10-30% (w/w), such as 10%, 11%, 12%, 13%, 14%, 15%, 16%, 17%, 18%, 19%, 20%, 21%, 22%, 23%, 24%, 25%, 26%, 27%, 28%, 29% or 30% (w/w) or any value in between.

In an embodiment, when an ionizable calixarene is used in RNA lipid nanoparticles (RNA-LNPs) to replace the traditional ionizable lipid (4 components: ionizable calixarene, a helper lipid, a sterol, and a PEG lipid), the mass of said sterol is 15-50% of the total mass of the delivery system.

In an embodiment of the 4-component system, said sterol is present in said delivery system at a mass fraction of 15-50% (w/w), such as 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% (w/w) or any value in between.

In an embodiment, when an ionizable calixarene is used in RNA lipid nanoparticles (RNA-LNPs) to replace the traditional ionizable lipid (4 components: ionizable calixarene, a helper lipid, a sterol, and a PEG lipid), the mass of said PEGylated lipid is 2-24%, more preferably 10-24% of the total mass of the delivery system.

In an embodiment of the 4-component system, said PEGylated lipid is present in said delivery system at a mass fraction of 2-24% (w/w), more preferably 10-24% (w/w), such as 10%, 11%, 12%, 13%, 14%, 15%, 16%, 17%, 18%, 19%, 20%, 21%, 22%, 23% or 24% (w/w) or any value in between.

The inventors further performed experiments to investigate the optimal ratio between the mass of the calixarene and the mass of the cargo to optimize encapsulation efficiency (see Example 2).

In a preferred embodiment, the delivery system of the current invention comprises a nucleic acid cargo, such as mRNA or saRNA.

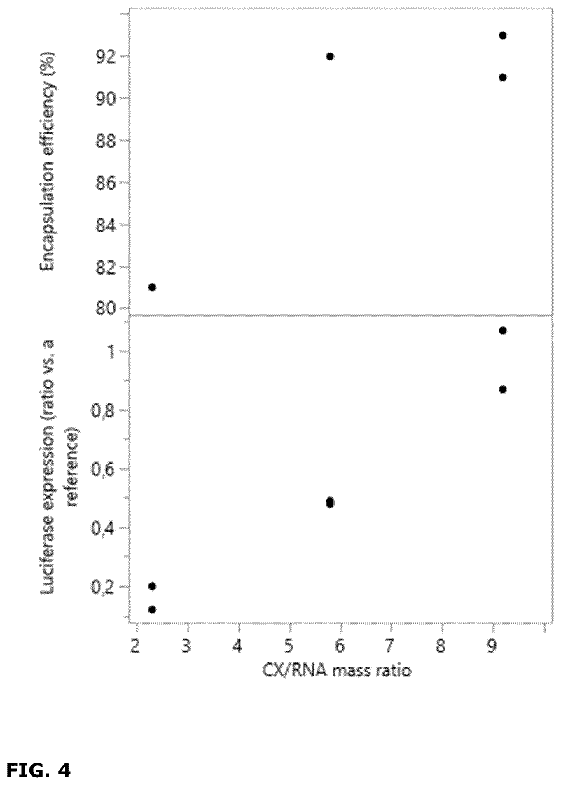

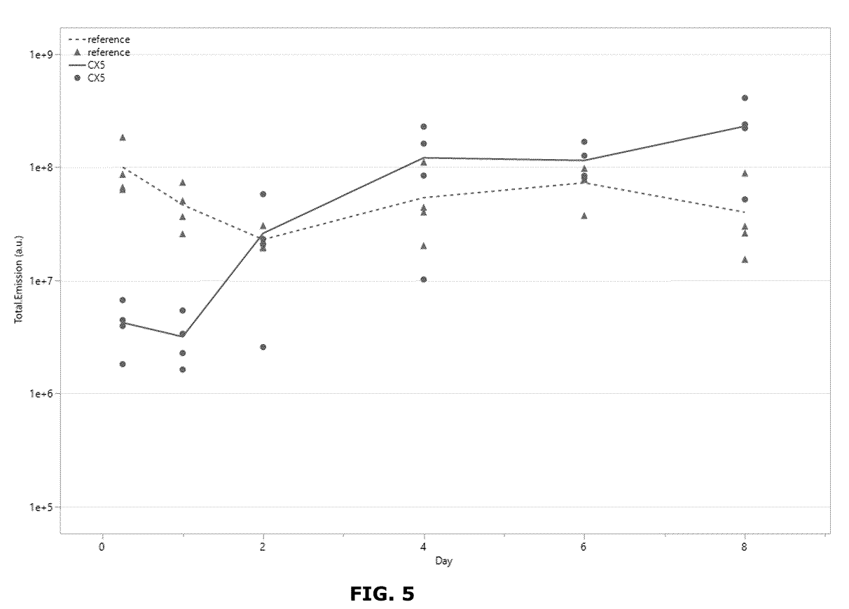

The inventors discovered an optimized mass ratio (Calixarene/saRNA) (see Example 2) that allowed to obtain stable nanoparticles with high encapsulation efficiency (>80%) (see FIG. 4) and adequate protein expression in vivo (see FIG. 5).

In an embodiment, said delivery system comprising an ionizable calixarene, a phospholipid, a sterol and a PEGylated lipid and wherein said cargo comprises a nucleic acid, wherein the mass of the ionizable calixarene is minimum the same mass as the cargo.

In an embodiment, said delivery system comprising an ionizable calixarene, a phospholipid, a sterol and a PEGylated lipid and wherein said cargo comprises a nucleic acid, wherein the mass of the ionizable calixarene is at most 15 times higher than the mass of the nucleic acid cargo.

In an embodiment, the delivery system comprises an ionizable calixarene, a phospholipid, a sterol and a PEGylated lipid and comprises a nucleic acid cargo, wherein the mass of the ionizable calixarene is 5, 6, 7, 8, 9, 10, 11, 12, 13, 14 or 15 times higher than the mass of the nucleic acid cargo.

In an embodiment, the delivery system comprises an ionizable calixarene, a phospholipid, a sterol and a PEGylated lipid and comprises a nucleic acid cargo, wherein the mass of the ionizable calixarene is between 5-6, 6-7, 7-8, 8-9, 9-10, 10-11, 11-12, 12-13, 13-14 or 14-15 times higher than the mass of the nucleic acid cargo.

As described above, saRNA (~10 000 nt) is larger than mRNA (~1000 nt) and has more secondary structure, making it more difficult to encapsulate and deliver. The inherent chemical and structural differences between mRNA and saRNA in terms of length, stability, and charge density suggests that LNP delivery formulations for saRNA may require conditions significantly different from those developed for mRNA delivery.

In a preferred embodiment, the delivery system comprises an ionizable calixarene, a phospholipid, a sterol and a PEGylated lipid and comprises an saRNA cargo, wherein the mass of the ionizable calixarene is at most 15 times higher, more preferably at most 10 times higher than the mass of the saRNA cargo.

In an embodiment, the delivery system comprises an ionizable calixarene, a phospholipid, a sterol and a PEGylated lipid and further comprises an saRNA cargo, wherein the mass of the ionizable calixarene is 5 to 15 times higher than the mass of the saRNA cargo.

In an embodiment, the delivery system comprises an ionizable calixarene, a phospholipid, a sterol and a PEGylated lipid and comprises an saRNA cargo, wherein the mass of the ionizable calixarene is 5, 6, 7, 8, 9, 10, 11, 12, 13, 14 or 15 times higher than the mass of the saRNA cargo.

In an embodiment, the delivery system comprises an ionizable calixarene, a phospholipid, a sterol and a PEGylated lipid and comprises an saRNA cargo, wherein the mass of the ionizable calixarene is between 5-6, 6-7, 7-8, 8-9, 9-10, 10-11, 11-12, 12-13, 13-14 or 14-15 times higher than the mass of the saRNA cargo.

It is worth mentioning that this optimized mass ratio is smaller in comparison to those needed (ca. 20 μg i-lipid/μg RNA) to reach similar properties with saRNA (encapsulation efficiency and in vitro protein expression) with well-known ionizable lipids used in the field (SM-102, ALC-0315).

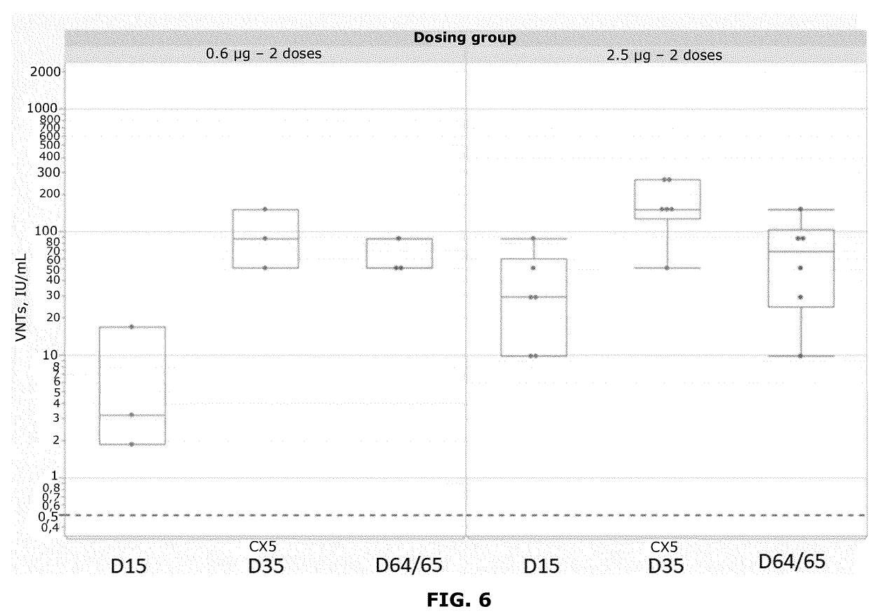

The inventors further preclinically validated a delivery system of the current invention comprising an ionizable calixarene (CX5), a phospholipid, a sterol and a PEGylated lipid. Mice were vaccinated following a 21 days prime-boost regimen using 0.6 μg or 2.5 μg of mRNA per dose. As shown in FIG. 6, the delivery system yielded strong VNTs (virus neutralization titers), well above the correlate of protection (0.5 IU/mL), using the two dosing regimens (0.6 or 2.5 μg). These results were accompanied without any adverse effect (loss of weight, impact on spleen, liver and kidneys weight, inflammation or an excessive reactogenic response) on the animals.

### 5-Component System

In an alternative embodiment, the current invention provides a delivery system wherein a cationic calixarene is incorporated as a fifth component into lipid nanoparticles (LNPs) made of an ionizable lipid, a helper lipid, a sterol, and a PEG lipid (5 components).

In a preferred embodiment, said cationic calixarene is a calix[4] arene.

As described above and referring to Formula (I), the calix[4] arene used in the current invention can comprises 4 identical B-groups. In another embodiment, the calix[4] arene used in the current invention can comprises 3 identical B-groups and one B-group which is different. In another embodiment, the calix[4] arene used in the current invention can comprises 2 identical B-groups and 2 other identical B-groups. The same holds true for the A-groups.

In an embodiment of the 5-component system, said calixarene is a calix[4] arene bearing 4 cationic head groups. In a further embodiment, said calixarene comprises 4 identical cationic head groups.

In an embodiment, one or more of said cationic head groups comprises at least one quaternary amine group. In an embodiment, each of said cationic head groups comprises at least one quaternary amine group. In an embodiment, one or more of said cationic head groups comprises more than one quaternary amine group.

The inventors demonstrated that these cationic calixarenes can be embedded into lipid nanoparticles encapsulating RNA (Table 3) and are made with:
Ionizable lipids: for instance DODAP, DLin-DMA, DLin-MC3-DMA, ALC-0315,

### SM-102;

Helper lipids: for instance DOPE, DOPC;
Sterol: for instance cholesterol;
and PEG lipids: for instance DMG-PEG2000, DSG-PEG2000.

As such, in an embodiment, when a cationic calixarene is incorporated as a fifth component into lipid nanoparticles (LNPs) made of an ionizable lipid, a helper lipid, a sterol, and a PEG lipid (5 components), the mass of said cationic calixarene is 0.1-60% (w/w), preferably 0.1-50% (w/w) of the total mass of the delivery system.

In an embodiment of the 5 component-system, said cationic calixarene is present in said delivery system at a mass fraction of 0.1-10% (w/w), 10-20% (w/w), 20-30% (ww/w), 30-40% (w/w), 40-50% (w/w) or 50-60% (w/w).

In an embodiment of the 5 component-system, said cationic calixarene is present in said delivery system at a mass fraction of 0.1-50% (w/w), 0.1-40%, 0.1-30%, 0.1-20% or 0.1-10% (w/w).

In an embodiment of the 5 component-system, said cationic calixarene is present in said delivery system at a mass fraction of 0.2-10% (w/w), such as 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5, 8%, 8.5%, 9%, 9.5%, 10% (w/w) or any value in between. In an embodiment, said cationic calixarene is present in said delivery system at a mass fraction between 0.1-10%, 10-20%, 20-30%, 30-40% or 40-50% (w/w). In an embodiment, said cationic calixarene is present in delivery system at a mass fraction between 0.1-1%, 1-2%, 2-3%, 3-4%, 4-5%, 5-6%, 6-7%, 7-8%, 8-9%, 9-10%, 10-11%, 11-12%, 12-13%, 13-14% or 14-15% (w/w).

In an embodiment, when a cationic calixarene is incorporated as a fifth component into lipid nanoparticles (LNPs) made of an ionizable lipid, a helper lipid, a sterol, and a PEG lipid (5 components), the mass of said phospholipid is 5-35%, more preferably 10-30% of the total mass of the delivery system.

In an embodiment of the 5-component system, said phospholipid is present in said delivery system at a mass fraction of 5-35% (w/w), more preferably 10-30% (w/w), such as 10%, 11%, 12%, 13%, 14%, 15%, 16%, 17%, 18%, 19%, 20%, 21%, 22%, 23%, 24%, 25%, 26%, 27%, 28%, 29% or 30% (w/w) or any value in between.

In an embodiment, when a cationic calixarene is incorporated as a fifth component into lipid nanoparticles (LNPs) made of an ionizable lipid, a helper lipid, a sterol, and a PEG lipid (5 components), the mass of said sterol is 15-50% of the total mass of the delivery system.

In an embodiment of the 5-component system, said sterol is present in said delivery system at a mass fraction of 15-50% (w/w), such as 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% (w/w) or any value in between.

In an embodiment, when a cationic calixarene is incorporated as a fifth component into lipid nanoparticles (LNPs) made of an ionizable lipid, a helper lipid, a sterol, and a PEG lipid (5 components), the mass of said PEGylated lipid is 2-24%, more preferably 10-24% of the total mass of the delivery system.

In an embodiment of the 5-component system, said PEGylated lipid is present in said delivery system at a mass fraction of 2-24% (w/w), more preferably 10-24% (w/w), such as 10%, 11%, 12%, 13%, 14%, 15%, 16%, 17%, 18%, 19%, 20%, 21%, 22%, 23% or 24% (w/w) or any value in between.

In an embodiment, when a cationic calixarene is incorporated as a fifth component into lipid nanoparticles (LNPs) made of an ionizable lipid, a helper lipid, a sterol, and a PEG lipid (5 components), the mass of said ionizable lipid is 15-50% of the total mass of the delivery system.

In an embodiment of the 5-component system, said ionizable lipid is present in said delivery system at a mass fraction of 15-50% (w/w), such as 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% (w/w) or any value in between.

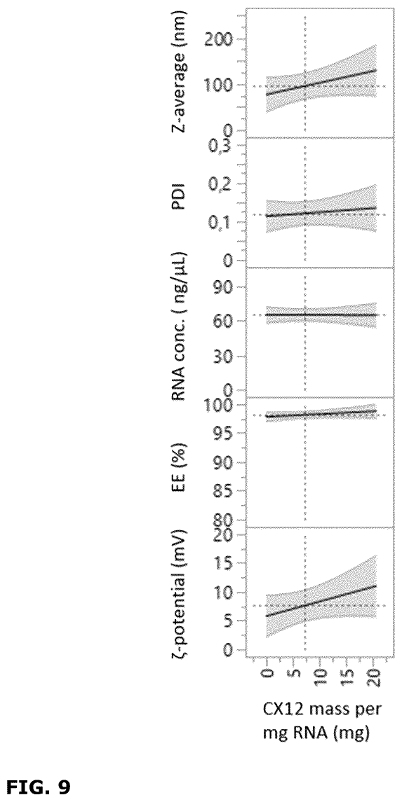

The inventors further performed experiments and showed that LNPs made with between 0.1 and 20.7 times more cationic calixarene than mRNA (mass ratio) were all found to be monodisperse and encapsulate more than 95% of the RNA. The addition of the cationic calixarene was found to increase both size (from 77 to 129 nm) and charge (from 5.8 to 11.0 mV) (see table 4 and FIG. 9, Example 3).

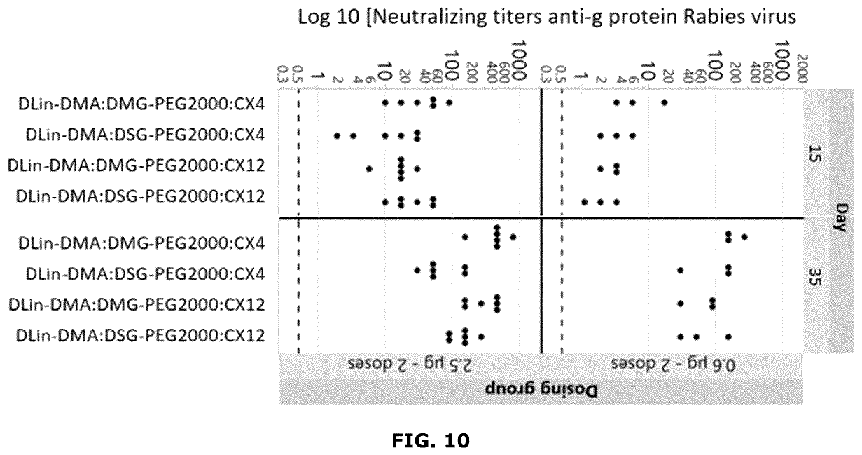

The inventors further demonstrated that cationic calixarenes combined with DLin-DMA as the ionizable lipid yields potent LNPs (Example 3). These results also suggest that the PEGylated lipid choice impacts the immune response: a difference as small as 4 additional carbons on the two alkyl chains of the PEGylated lipid can significantly decrease the immune potency of an otherwise identical LNP (see FIG. 10, Example 3).

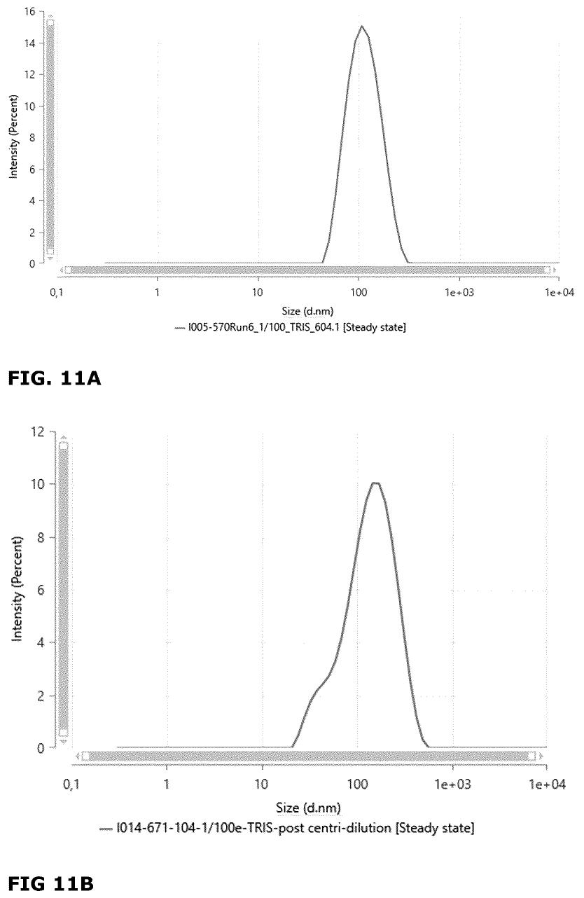

The inventors further demonstrated that the 5-component system can be used to efficiently encapsulate saRNAs. The resulting LNPs was found monodisperse with a Z-average of 120 nm (FIG. 11A) and encapsulated 76.6% of the saRNA. This result is particularly interesting as common ionizable lipids are difficult to use to encapsulate saRNA. This was demonstrated with an saRNA of similar length and encoding for an undisclosed gene of interest. The saRNA was formulated using a SM-102: DSPC: cholesterol:DMG-PEG2000 (50:10:38.5:1.5 molar ratio). The resulting LNP was found polydisperse (FIG. 11B).

According to the invention, inclusion of buffered agents and solvents can be further present in the delivery system and/or aid with the production thereof. For example, for the fabrication of the delivery system, preferred solvent is ethanol for the lipid phase and acidic aqueous buffer for the aqueous phase.

In embodiments, the preferred buffers are citrate and acetate buffers with a ranging pH and concentrations. In specific embodiments the pH of the buffers are between pH 3-6 and the concentration is ranging between 1 and 100 mM.

In embodiments, the preferred buffers are citrate and acetate buffers with a ranging pH and concentrations. In specific embodiments the pH of the buffers are between pH 3-5.5 and the concentration is ranging between 10-50 mM. However, none of the example buffers given above are limiting as various buffers known in the field that are used for the formation of the delivery systems can be used and that is obvious to a skilled person. Not limiting but preferred method of fabrication is microfluidic mixing.

The (mean) diameter of the delivery system can be quantified by any means known from the state of the art, such as quasi-electric light scattering (QELS), dynamic light scattering (DLS), Nanoparticle Tracking Analysis (NTA) and by imaging methods (such as, scanning electron microscopy (SEM), transmission electron microscopy (TEM) and cryo-(TEM)). In an embodiment, DLS allows to determine the average particle size and polydispersity index (PDI, a measure of particle size distribution), while NTA allows to determine the mean, mode, and span of the particle population.

In an embodiment, the delivery system comprises a monodispersed population. As such, in a preferred embodiment, the delivery system has a PDI below 0.25, preferably below 0.2.

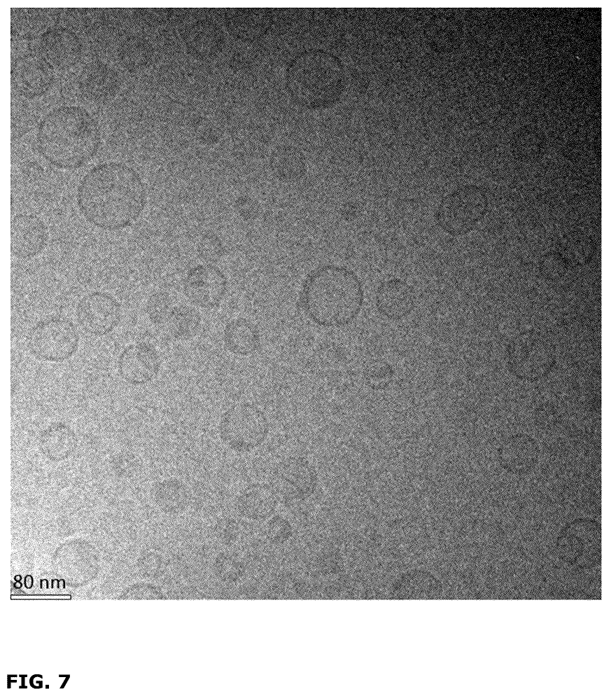

FIG. 7 shows a delivery system according to the current invention, more specifically an LNP comprising an ionizable calixarene, a phospholipid, a sterol and a PEGylated lipid (CX5: DOPE: cholesterol:DMG-PEG2000 nanoparticles encapsulating mRNa-FLuc) which were imaged using cryo-TEM. The LNPs as imaged are monodisperse, with the smallest and largest LNPs around 40 nm and 80 nm respectively, which is in good agreement with DLS measurements (Z-average of ca. 50 nm).

In an embodiment, advanced mathematical analyses (e.g. CUMULANT analysis) can be used to estimate the mean size and PDI of the delivery system of the current disclosure. Besides influencing the biodistribution, the diameter also influences the loading capacity of the delivery system.

In an embodiment, the mean diameter of the delivery system is between 10 nm and 10 000 nm, more preferably between 10 nm and 9000 nm, more preferably between 10 nm and 8000 nm, more preferably between 10 nm and 7000 nm, more preferably between 10 nm and 6000 nm, more preferably between 10 nm and 5000 nm, more preferably between 10 nm and 4000 nm, more preferably between 10 nm and 3000 nm, more preferably between 10 nm and 2000 nm, more preferably the mean diameter of the delivery system is between 10 and 1000 nm, preferably between 50 nm and 200 nm. In an embodiment, the mean diameter of the delivery system is at least 10 nm, preferably at least 20 nm, more preferably at least 25 nm, more preferably at least 30 nm, more preferably at least 35 nm, more preferably at least 40 nm, more preferably at least 45 nm, more preferably at least 50 nm. In an embodiment, the mean diameter of the delivery system is at most 900 nm, more preferably at most 800 nm, more preferably at most 700 nm, more preferably at most 600 nm, more preferably at most 500 nm, more preferably at most 400 nm, more preferably at most 300 nm, more preferably at most 200 nm.

In an embodiment, the mean diameter of the delivery system is between 10 nm and 200 nm, between 10 nm and 190 nm, between 10 nm and 180 nm, between 10 nm and 170 nm, between 10 nm and 160 nm, between 10 nm and 150 nm, between 10 nm and 140 nm, between 10 nm and 130 nm, between 10 nm and 120 nm, between 10 nm and 110 nm, between 10 nm and 100 nm, between 10 nm and 90 nm, between 10 nm and 80 nm, between 10 nm and 70 nm, between 10 nm and 60 nm, between 10 nm and 50 nm, between 10 nm and 40 nm, between 10 nm and 30 nm, between 10 nm and 20 nm, between 20 nm and 200 nm, between 20 nm and 190 nm, between 20 nm and 180 nm, between 20 nm and 170 nm, between 20 nm and 160 nm, between 20 nm and 150 nm, between 20 nm and 140 nm, between 20 nm and 130 nm, between 20 nm and 120 nm, between 20 nm and 110 nm, between 20 nm and 100 nm, between 20 nm and 90 nm, between 20 nm and 80 nm, between 20 nm and 70 nm, between 20 nm and 60 nm, between 20 nm and 50 nm, between 20 nm and 40 nm, between 20 nm and 30 nm, between 30 nm and 200 nm, between 30 nm and 190 nm, between 30 nm and 180 nm, between 30 nm and 170 nm, between 30 nm and 160 nm, between 30 nm and 150 nm, between 30 nm and 140 nm, between 30 nm and 130 nm, between 30 nm and 120 nm, between 30 nm and 110 nm, between 30 nm and 100 nm, between 30 nm and 90 nm, between 30 nm and 80 nm, between 30 nm and 70 nm, between 30 nm and 60 nm, between 30 nm and 50 nm, between 30 nm and 40 nm, between 40 nm and 200 nm, between 40 nm and 190 nm, between 40 nm and 180 nm, between 40 nm and 170 nm, between 40 nm and 160 nm, between 40 nm and 150 nm, between 40 nm and 140 nm, between 40 nm and 130 nm, between 40 nm and 120 nm, between 40 nm and 110 nm, between 40 nm and 100 nm, between 40 nm and 90 nm, between 40 nm and 80 nm, between 40 nm and 70 nm, between 40 nm and 60 nm, between 40 nm and 50 nm, between 50 nm and 200 nm, between 50 nm and 190 nm, between 50 nm and 180 nm, between 50 nm and 170 nm, between 50 nm and 160 nm, between 50 nm and 150 nm, between 50 nm and 140 nm, between 50 nm and 130 nm, between 50 nm and 120 nm, between 50 nm and 110 nm, between 50 nm and 100 nm, between 50 nm and 90 nm, between 50 nm and 80 nm, between 50 nm and 70 nm, between 50 nm and 60 nm, between 60 nm and 200 nm, between 60 nm and 190 nm, between 60 nm and 180 nm, between 60 nm and 170 nm, between 60 nm and 160 nm, between 60 nm and 150 nm, between 60 nm and 140 nm, between 60 nm and 130 nm, between 60 nm and 120 nm, between 60 nm and 110 nm, between 60 nm and 100 nm, between 60 nm and 90 nm, between 60 nm and 80 nm, between 60 and 70 nm, between 70 nm and 200 nm, between 70 nm and 190 nm, between 70 nm and 180 nm, between 70 nm and 170 nm, between 70 nm and 160 nm, between 70 nm and 150 nm, between 70 nm and 140 nm, between 70 nm and 130 nm, between 70 nm and 120 nm, between 70 nm and 110 nm, between 70 nm and 100 nm, between 70 nm and 90 nm, between 70 nm and 80 nm, between 80 nm and 200 nm, between 80 nm and 190 nm, between 80 nm and 180 nm, between 80 nm and 170 nm, between 80 nm and 160 nm, between 80 nm and 150 nm, between 80 nm and 140 nm, between 80 nm and 130 nm, between 80 nm and 120 nm, between 80 nm and 110 nm, between 80 nm and 100 nm, between 80 nm and 90 nm, between 90 nm and 200 nm, between 90 nm and 190 nm, between 90 nm and 180 nm, between 90 nm and 170 nm, between 90 nm and 160 nm, between 90 and 150 nm, between 90 nm and 140 nm, between 90 nm and 130 nm, between 90 nm and 120 nm, between 90 nm and 110 nm, between 90 nm and 100 nm, between 100 nm and 200 nm, between 100 nm and 190 nm, between 100 nm and 180 nm, between 100 nm and 170 nm, between 100 nm and 160 nm, between 100 and 150 nm, between 100 nm and 140 nm, between 100 nm and 130 nm, between 100 nm and 120 nm, between 100 nm and 110 nm, between 110 nm and 200 nm, between 110 nm and 190 nm, between 110 nm and 180 nm, between 110 nm and 170 nm, between 110 nm and 160 nm, between 110 and 150 nm, between 110 nm and 140 nm, between 110 nm and 130 nm, between 110 nm and 120 nm, between 120 nm and 200 nm, between 120 nm and 190 nm, between 120 nm and 180 nm, between 120 nm and 170 nm, between 120 nm and 160 nm, between 120 nm and 150 nm, between 120 nm and 140 nm, between 120 nm and 130 nm, between 130 nm and 200 nm, between 130 nm and 190 nm, between 130 nm and 180 nm, between 130 nm and 170 nm, between 130 nm and 160 nm, between 130 nm and 150 nm, between 130 nm and 140 nm, between 140 nm and 200 nm, between 140 nm and 190 nm, between 140 nm and 180 nm, between 140 nm and 170 nm, between 140 nm and 160 nm, between 140 nm and 150 nm, between 150 nm and 200 nm, between 150 nm and 190 nm, between 150 nm and 180 nm, between 150 nm and 170 nm, between 150 nm and 160 nm, between 160 nm and 200 nm, between 160 nm and 190 nm, between 160 nm and 180 nm, between 160 nm and 170 nm, between 170 nm and 200 nm, between 170 nm and 190 nm, between 170 nm and 180 nm, between 170 nm and 180 nm, between 180 nm and 190 nm, between 190 nm and 200 nm.

In an embodiment, the mean diameter of the delivery system is between 200 nm and 1000 nm, between 200 nm and 950 nm, between 200 nm and 900 nm, between 200 nm and 850 nm, between 200 nm and 800 nm, between 200 nm and 750 nm, between 200 nm and 700 nm, between 200 nm and 650 nm, between 200 nm and 600 nm, between 200 nm and 550 nm, between 200 nm and 500 nm, between 200 nm and 450 nm, between 200 nm and 400 nm, between 200 nm and 350 nm, between 200 nm and 300 nm, between 200 nm and 250 nm, between 250 nm and 1000 nm, between 250 nm and 950 nm, between 250 nm and 900 nm, between 250 nm and 850 nm, between 250 nm and 800 nm, between 250 nm and 750 nm, between 250 nm and 700 nm, between 250 nm and 650 nm, between 250 nm and 600 nm, between 250 nm and 550 nm, between 250 nm and 500 nm, between 250 nm and 450 nm, between 250 nm and 400 nm, between 250 nm and 350 nm, between 250 nm and 300 nm, between 300 nm and 1000 nm, between 300 nm and 950 nm, between 300 nm and 900 nm, between 300 nm and 850 nm, between 300 nm and 800 nm, between 300 nm and 750 nm, between 300 nm and 700 nm, between 300 nm and 650 nm, between 300 nm and 600 nm, between 300 nm and 550 nm, between 300 nm and 500 nm, between 300 nm and 450 nm, between 300 nm and 400 nm, between 300 nm and 350 nm, between 350 nm and 1000 nm, between 350 nm and 950 nm, between 350 nm and 900 nm, between 350 nm and 850 nm, between 350 nm and 800 nm, between 350 nm and 750 nm, between 350 nm and 700 nm, between 350 nm and 650 nm, between 350 nm and 600 nm, between 350 nm and 550 nm, between 350 nm and 500 nm, between 350 nm and 450 nm, between 350 nm and 400 nm, between 400 nm and 1000 nm, between 400 nm and 950 nm, between 400 nm and 900 nm, between 400 nm and 850 nm, between 400 nm and 800 nm, between 400 nm and 750 nm, between 400 nm and 700 nm, between 400 nm and 650 nm between 400 nm and 600 nm, between 400 nm and 550 nm, between 400 and 500 nm, between 400 nm and 450 nm, between 450 nm and 1000 nm, between 450 nm and 950 nm, between 450 nm and 900 nm, between 450 nm and 850 nm, between 450 nm and 800 nm, between 450 nm and 750 nm, between 450 nm and 700 nm, between 450 nm and 650 nm, between 450 nm and 600 nm, between 450 nm and 550 nm, between 450 nm and 500 nm, between 500 nm and 1000 nm, between 500 nm and 950 nm, between 500 nm and 900 nm, between 500 nm and 850 nm, between 500 nm and 800 nm, between 500 nm and 750 nm, between 500 nm and 700 nm, between 500 nm and 650 nm, between 500 and 600 nm, between 500 nm and 550 nm, between 550 nm and 1000 nm, between 550 nm and 950 nm, between 550 nm and 900 nm, between 550 nm and 850 nm, between 550 nm and 800 nm, between 550 nm and 750 nm, between 550 nm and 700 nm, between 550 nm and 650 nm, between 550 and 600 nm, between 600 nm and 1000 nm, between 600 nm and 950 nm, between 600 nm and 900 nm, between 600 nm and 850 nm, between 600 nm and 800 nm, between 600 nm and 750 nm, between 600 nm and 700 nm, between 600 nm and 650 nm, between 650 nm and 1000 nm, between 650 and 950 nm, between 650 nm and 900 nm, between 650 nm and 850 nm, between 650 nm and 800 nm, between 650 nm and 750 nm, between 650 nm and 700 nm, between 700 nm and 1000 nm, between 700 nm and 950 nm, between 700 nm and 900 nm, between 700 nm and 850 nm, between 700 nm and 800 nm, between 700 nm and 750 nm, between 750 nm and 1000 nm, between 750 nm and 950 nm, between 750 nm and 900 nm, between 750 nm and 850 nm, between 750 nm and 800 nm, between 800 nm and 1000 nm, between 800 nm and 950 nm, between 800 nm and 900 nm, between 800 nm and 850 nm, between 850 nm and 950 nm, between 850 nm and 900 nm, between 900 nm and 950 nm, between 950 nm and 1000 nm.

The cargo to be delivered using the calixarene-based delivery system can be selected from the group consisting of a nucleic acid, a protein, a chemical substance, a polysaccharide, and combinations thereof. Preferably, the cargo is a nucleic acid, more preferably RNA or DNA. The cargo may include therapeutic agents, such as gene therapies, siRNA, mRNA, CRISPR/Cas9 components, proteins, enzymes, antibodies, small molecules, or other chemical substances, as well as imaging agents or contrast agents for diagnostic applications. The cargo can be encapsulated within the delivery system or be associated with the system through electrostatic interactions, covalent bonding, or other means of attachment.

The cargo can be encapsulated within the delivery system or be associated with the system through ion pairing interactions, covalent bonding, or other means of attachment.

In some embodiments, the cargo is selected from long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), Trans amplifying mRNA, RNA oligonucleotides, antisense oligonucleotides, small interfering RNA (SIRNA), small hairpin RNA (shRNA), antisense RNA (asRNA),

CRISPR/Cas9 guide RNAs (gRNA), riboswitches, im-munostimulating RNA (isRNA), ribozymes, aptamers, ribosomal RNA (rRNA), transfer RNA (TRNA), viral RNA (vRNA), retroviral RNA or replicon RNA, small nuclear RNA (snRNA), small nucleolar RNA (snoRNA), microRNA (miRNA), and a Piwi-interacting RNA (piRNA).

In an embodiment, the cargo is a micro-RNA, saRNA, circRNA or mRNA.

In some embodiments, the cargo comprises modified RNA molecules. In some embodiments, the modification of RNA molecule comprises chemical modifications comprising backbone modifications as well as sugar modifications or base modifications. In this context, a modified RNA molecule as defined herein comprises nucleotide analogues/modifications, e.g. backbone modifications, sugar modifications or base modifications. A backbone modification in connection with the present disclosure is a modification, in which phosphates of the backbone of the nucleotides contained in an RNA molecule are chemically modified. A sugar modification in connection with the present disclosure is a chemical modification of the sugar of the nucleotides of the RNA molecule. Furthermore, a base modification in connection with the present disclosure is a chemical modification of the base moiety of the nucleotides of the RNA molecule. In this context, nucleotide analogues or modifications are selected from nucleotide analogues, which are applicable for transcription and/or translation. In further embodiments, the modified RNA comprises nucleoside modifications selected from 6-aza-cytidine, 2-thio-cytidine, a-thio-cytidine, pseudo-iso-cytidine, 5-aminoallyl-uridine, 5-iodo-uridine, N1-methyl-pseudouridine, 5,6-dihydrouridine, a-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine.

In an embodiment, the cargo is self-amplifying RNA (saRNA).

It was found that the delivery system is also suited to be used in combination with RNA of a large size, such as self-amplifying RNA (saRNA). Since saRNA is larger than conventional mRNA, lipid nanoparticles described in the state of the art often function improperly as they result in poor encapsulation, or sub-optimal in vivo delivery. A delivery system as currently defined can solve this.

By preference, the size of the (sa) RNA can be between 500 and 50 000 nucleotides (Nt), preferably between 1000 and 40 000 Nt, more preferably between 5000 and 30 000, or between 8000 and 16 000 Nt.

The self-replicative nature of the mRNA constructs is based on the genomic RNA of RNA viruses, but lack the genes encoding one or more structural proteins. The self-replicating RNA molecules are capable of being translated to produce non-structural proteins of the RNA virus and heterologous proteins encoded by the self-replicating RNA. Self-replicating RNA molecules are designed so that the self-replicating RNA molecule cannot induce production of infectious viral particles. One suitable system for achieving self-replication is to use an alphavirus-based RNA replicon. These +−stranded replicons are translated after delivery to a cell to give of a replicase (or replicase-transcriptase). The replicase is translated as a polyprotein which auto-cleaves to provide a replication complex which creates genomic—strand copies of the +−strand delivered RNA. These—−strand transcripts can themselves be transcribed to give further copies of the +−stranded parent RNA and also to give a subgenomic transcript which encodes the desired gene product. Translation of the subgenomic transcript thus leads to in situ expression of the desired gene product by the cell. Suitable alphavirus replicons can use a replicase from a Sindbis virus, a Semliki Forest Virus, an eastern equine encephalitis virus, a Venezuelan Equine Encephalitis Virus, etc. A preferred self-replicating RNA molecule encodes (i) a RNA-dependent RNA polymerase which can transcribe RNA from the self-replicating RNA molecule and (ii) protein/peptide of interest. The polymerase can be an alphavirus replicase e.g., comprising alphavirus protein nsP4.

In embodiments, the pharmaceutical compositions comprising the delivery system may be lyophilized or stabilized in a dry form. “Lyophilizing” in this document refers to freeze-drying a liquid or pre-lyophilization formulation. Freeze-drying is performed by freezing the formulation and then subliming ice from the frozen content at a temperature suitable for primary drying. Under this condition the product temperature is below the collapse temperature of the formulation. A secondary drying stage may then be carried out, which produces a suitable lyophilized cake. Lyophilization is commonly used in the production of pharmaceutical compounds to increase the stability of the Active Pharmaceutical Ingredient (API) by removing solvents. Lyophilization offers many advantages as it allows the processing and development of pharmaceutical compounds, otherwise unstable in solution, hence improving their shelf life. This technique can facilitate development, usage, distribution and commercialization of new drugs.

### Method Of Use And Methods Of Treatment

The present invention also provides a method for delivering one or more cargos to a subject using the calixarene-based delivery system. The method includes the following steps:

Preparation of the delivery system: The delivery system components, including calixarene, phospholipid, additional lipid such as sterol, optionally a PEGylated lipid, and optionally ionizable lipid, are combined and formulated into a delivery system. The cargo to be delivered is incorporated into the delivery system, either by encapsulation, association through ion pairing interactions, or other means of attachment. The cargo can be incorporated during or post-combination of the components of the delivery system. Hereafter delivery system refers to delivery system with cargo.

Administration of the delivery system: The prepared delivery system is administered to a subject, such as a patient in need of the cargo's therapeutic effects or diagnostic information. The administration can be carried out using various routes, such as intravenous, intramuscular, subcutaneous, oral, inhalation, or other suitable routes, depending on the target tissue, the cargo, and the desired therapeutic or diagnostic outcome. The system as detailed herein may be formulated for any available delivery route, including an oral, mucosal (e.g., nasal, sublingual, vaginal, buccal, or rectal), parenteral (e.g., intraarticular, intravenous, intraperitoneal, intramuscular, intradermal or subcutaneous injection), topical or transdermal delivery form. Formulations suitable for parenteral administration include aqueous and non-aqueous, isotonic sterile injection solutions or suspensions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally. For the injection, not limiting but preferred buffer is TRIS buffer with sucrose.

The delivery system may be prepared in unit dose form. In some embodiments a unit dose may have a volume of between about 0.1-1.0 ml, e.g. about 0.5 ml. The pharmaceutical compositions or vaccines can be presented in unit-dose or multi-dose sealed containers, such as ampoules and vials. Injection solutions and suspensions can be prepared from sterile powders, granules, and tablets. Cells transfected with the pharmaceutical composition of the invention can also be administered intravenously or parenterally.

The delivery system can be administered as a single dose or as a multi-dose, requiring a series of two or more doses, administered within a pre-defined timespan. Such timespan may be a week, two weeks, three weeks, four weeks, five weeks, six weeks, seven weeks, 8 weeks, 9 weeks, 10 weeks, 11 weeks up until one year.

Delivery of cargo to target cells: Once the delivery system is administered, it can facilitate the transport of the cargo to the target cells. The calixarene-based delivery system may enhance cellular uptake, endosomal escape, and release of the cargo into the cytoplasm or other intracellular compartments, depending on the cargo and the target cells.

Evaluation of therapeutic or diagnostic effects: After the cargo has been delivered to the target cells, the therapeutic or diagnostic effects can be assessed. This may include monitoring changes in gene expression, protein levels, cellular function, disease progression, or other relevant outcomes. In the case of diagnostic applications, imaging or other techniques may be employed to visualize the distribution and effects of the cargo.

In a further aspect, the invention relates to a (pharmaceutical) composition comprising a delivery system comprising cargo as described above. Further, the invention relates to a method of treating a disease or disorder using the (pharmaceutical) composition. In some embodiments, the composition is for use as a human or veterinary medicament.

In an embodiment, said pharmaceutical composition is used in gene therapy. Gene therapy can include protein replacement strategies (for instance by means of DNA or mRNA delivery) or gene silencing (for instance by means of siRNA or miRNA delivery). In a preferred embodiment, said pharmaceutical composition is a vaccine or can be used to immunize or vaccinate subjects.

In an embodiment, the present disclosure provides RNA (e.g., mRNA) vaccines that include at least one RNA (e.g., mRNA) polynucleotide having an open reading frame encoding at least one antigenic polypeptide or an immunogenic fragment thereof (e.g., an immunogenic fragment capable of inducing an immune response to the antigenic polypeptide). While not wishing to be bound by theory, it is believed that the RNA (e.g., mRNA) vaccines, for example, as mRNA polynucleotides, are better designed to produce the appropriate protein conformation upon translation, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Pharmaceutical compositions comprise an immunologically effective amount of polynucleotides, as well as any other components, as needed. By ‘immunologically effective amount’, it is meant that the administration of that amount to a subject, either in a single dose or as part of a series, is effective for treatment or prophylaxis. This amount varies depending upon the health and physical condition of the subject to be treated, age, the taxonomic group of subject to be treated (e.g. non-human primate, primate, etc.), the capacity of the subject's immune system to synthesize antibodies, the degree of protection desired, the formulation of the vaccine, the treating doctor's assessment of the medical situation, and other relevant factors. It is expected that the amount will fall in a relatively broad range that can be determined through routine trials. The RNA content of the pharmaceutical compositions described herein will generally be expressed in terms of the amount of RNA per dose. In an embodiment, said dose has≤about 100 μg RNA (e.g. from 10-100 μg, such as about 10 μg, 25 μg, 50 μg, 75 μg or 100 μg), but expression can be seen at much lower levels, e.g. ≤1 μg/dose, ≤100 ng/dose, ≤10 ng/dose, ≤1 ng/dose, etc.

In a preferred embodiment, the polynucleotide in the pharmaceutical composition encodes an antigen, preferably an antigen linked to an infectious disease or agent.

In a specific embodiment, the antigen is a target-specific antigen which can be a tumor antigen, or a bacterial, viral or fungal antigen. Said target-specific antigen can be derived from either one of: total mRNA isolated from (a) target cell(s), one or more specific target mRNA molecules, protein lysates of (a) target cell(s), specific proteins from (a) target cell(s), or a synthetic target-specific peptide or protein and synthetic mRNA or DNA encoding a target-specific antigen or its derived peptides. To avoid any misunderstanding, the pharmaceutical compositions as disclosed herein may comprise a single mRNA molecule, or they may comprise multiple mRNA molecules, such as a combination of one or more mRNA molecules encoding immune modulating proteins and/or one or more mRNA molecules encoding antigen- and/or disease-specific proteins.

In an embodiment, said polynucleotides encode an immunogen. The immunogen may elicit an immune response against a bacterium, a virus, a fungus, a parasite, an allergen, or a tumor antigen. The immune response may comprise an antibody response (usually including IgG) and/or a cell-mediated immune response. The immunogen can for instance be a surface polypeptide (e.g. an adhesin, a hemagglutinin, an envelope glycoprotein, a spike glycoprotein, etc), an internal protein (e.g. a nucleoprotein) or any combination of any of the foregoing.

In some examples the immunogen elicits an immune response against one of these bacteria:
Neisseria meningitidis: useful immunogens include, but are not limited to, membrane proteins such as adhesins, autotransporters, toxins, iron acquisition proteins, and factor H binding protein.
Streptococcus pneumoniae: useful immunogens include, but are not limited to, the RrgB pilus subunit, the beta-N-acetyl-hexosaminidase precursor (spr0057), spr0096, General stress protein GSP-781 (spr2021, SP2216), serine/threonine kinase StkP (SP 1732), and pneumococcal surface adhesin PsaA.
Streptococcus pyogenes.
Moraxella catarrhalis.
Bordetella pertussis: Useful pertussis immunogens include, but are not limited to, pertussis toxin or toxoid (PT), filamentous haemagglutinin (FHA), pertactin, and agglutinogens 2 and 3.
Staphylococcus aureus: Useful immunogens include, but are not limited to a hemolysin, esxA, esxB, ferrichrome-binding protein (sta006) and/or the sta011 lipoprotein.
Clostridium tetani: the typical immunogen is tetanus toxoid.
Corynebacterium diphtheria: the typical immunogen is diphtheria toxoid.
Haemophilus influenzae
Pseudomonas aeruginosa
Streptococcus agalactiae
Chlamydia trachomatis: Useful immunogens include, but are not limited to, PepA, LcrE, ArtJ, Dnak, CT398, OmpH-like, L7/L12, OmcA, AtoS, CT547, Eno, HtrA and MurG.
Chlamydia pneumonia
Helicobacter pylori: Useful immunogens include, but are not limited to, CagA, VacA, NAP, and/or urease.
Escherichia coli: Useful immunogens include, but are not limited to, immunogens derived from enterotoxigenic E. coli (ETEC), enteroaggregative E. coli (EaggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC). A useful immunogen for several E. coli types is AcfD.
Bacillus anthracis
Yersinia pestis
Staphylococcus epidermis
Clostridium perfringens or Clostridium botulinum
Legionella pneumophila
Coxiella burwetii. Brucella, such as B.abortus, B.canis, B.melitensis, B.neotomae, B.ovis, B.suis, B.pinnipediae.
Francisella, such as F.novicida, F.philomiragia, F.tularensis.
Neisseria gonorrhoea
Treponema pallidum
Haemophilus ducreyi
Enterococcus faecalis or Enterococcus faecium
Staphylococcus saprophyticus
Yersinia enterocolitica
Mycobacterium tuberculosis
Rickettsia
Listeria monocytogenes
Vibrio cholera
Salmonella typhi
Borrelia burgdorferi
Porphyromonas gingivalis
Klebsiella

In some examples the immunogen elicits an immune response against one of these viruses:
Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g. H1, H2, H3, H4, H5, H6, H7, H8, H9, H10, H11, H12, H13, H14, H15 or H16.
Paramyxoviridae viruses: Viral immunogens include, but are not limited to, those derived from Pneumoviruses (e.g. respiratory syncytial virus, RSV), Rubulaviruses (e.g. mumps virus), Paramyxoviruses (e.g. parainfluenza virus), Metapneumoviruses and Morbilliviruses (e.g. measles).
Poxviridae: Viral immunogens include, but are not limited to, those derived from Orthopoxvirus such as Variola vera, including but not limited to, Variola major and Variola minor.
Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.
Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.
Heparnavirus: Viral immunogens include, but are not limited to, those derived from a Heparnavirus, such as hepatitis A virus (HAV).
Filovirus: Viral immunogens include, but are not limited to, those derived from a filovirus, such as an Ebola virus (including a Zaire, Ivory Coast, Reston or Sudan ebolavirus) or a Marburg virus.
Togavirus: Viral immunogens include, but are not limited to, those derived from a Togavirus, such as a Rubivirus, an Alphavirus, or an Arterivirus. This includes rubella virus.
Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.
Pestivirus: Viral immunogens include, but are not limited to, those derived from a Pestivirus, such as Bovine viral diarrhea (BVDV), Classical swine fever (CSFV) or Border disease (BDV).
Hepadnavirus: Viral immunogens include, but are not limited to, those derived from a Hepadnavirus, such as Hepatitis B virus. A composition can include hepatitis B virus surface antigen (HbsAg).
Other hepatitis viruses: A composition can include an immunogen from a hepatitis C virus, delta hepatitis virus, hepatitis E virus, or hepatitis G virus.
Rhabdovirus: Viral immunogens include, but are not limited to, those derived from a Rhabdovirus, such as a Lyssavirus (e.g. a Rabies virus) and Vesiculovirus (VSV).
Caliciviridae: Viral immunogens include, but are not limited to, those derived from Calciviridae, such as Norwalk virus (Norovirus), and Norwalk-like Viruses, such as Hawaii Virus and Snow Mountain Virus.
Coronavirus: Viral immunogens include, but are not limited to, those derived from a SARS coronavirus, avian infectious bronchitis (IBV), Mouse hepatitis virus (MHV), and Porcine transmissible gastroenteritis virus (TGEV). The coronavirus immunogen may be a spike polypeptide.
Retrovirus: Viral immunogens include, but are not limited to, those derived from an Oncovirus, a Lentivirus (e.g. HIV-1 or HIV-2) or a Spumavirus.
Reovirus: Viral immunogens include, but are not limited to, those derived from an Orthoreovirus, a Rotavirus, an Orbivirus, or a Coltivirus.
Parvovirus: Viral immunogens include, but are not limited to, those derived from Parvovirus B19.
Herpesvirus: Viral immunogens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).
Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.
Adenovirus: Viral immunogens include those derived from adenovirus serotype 36 (Ad-36).

Fungal immunogens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/orTrichophyton faviforme; or fromAspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glablata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumonia, Microsporidia, Encephalitozoon spp.,Septata intestinalis andEnterocytozoon bieneusi; the less common areBrachiola spp,Microsporidium spp., Nosema spp.,Pleistophora spp., Trachipleistophora spp., Vittaforma sppParacoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp.,Wangiella spp., Sporothrix spp., Basidiobolus spp., Conidiobolus spp., Rhizopus spp, Mucor spp, Absidia spp, Mortierella spp, Cunninghanlella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In some examples the immunogen elicits an immune response against a parasite from the Plasmodium genus, such as P.falciparum, P.vivax, P.malariae or P.ovale.

Thus the disclosure may be used for immunising against malaria. In some examples the immunogen elicits an immune response against a parasite from the Caligidae family, particularly those from the Lepeophtheirus and Caligus genera e.g. sea lice such as Lepeophtheirus salmonis or Caligus rogercresseyi.

In some examples the immunogen elicits an immune response against: pollen allergens (tree-, herb, weed-, and grass pollen allergens); insect or arachnid allergens (inhalant, saliva and venom allergens,e.g. mite allergens, cockroach and midges allergens, hymenopthera venom allergens); animal hair and dandruff allergens (from e.g. dog, cat, horse, rat, mouse,etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and flea se.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some examples the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with,e.g., bladder cancer), HLA-A2-R1701, beta catenin (associated with, e.g., melanoma), TCR (associated with,e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with,e.g., Hodgkin's disease), proteinase 3 (associated with, e.g., chronic myelogenous leukemia), WT 1 (associated with, e.g., various leukemias), carbonic anhydrase (associated with,e.g., renal cancer), aldolase A (associated with,e.g., lung cancer), PRAME (associated with,e.g., melanoma), HER-2/neu (associated with,e.g., breast, colon, lung and ovarian cancer), mammaglobin, alpha-fetoprotein (associated with,e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with, e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

In an embodiment, the delivery system can be used for efficiently delivering cancer vaccines to the target site in the body.

In an embodiment, the pharmaceutical composition comprises one polynucleotide. In an alternative embodiment, the pharmaceutical composition comprises more than one polynucleotide.

The disclosure also provides a delivery device (e.g. syringe, nebuliser, sprayer, inhaler, dermal patch,etc.) containing a pharmaceutical composition as disclosed herein. This device can be used to administer the pharmaceutical composition to a subject.

Also disclosed herein are methods of treatment. More particularly, disclosed herein are pharmaceutical compositions or delivery systems as described herein for use as a human or veterinary medicament. Even more particularly, the above pharmaceutical compositions of the disclosure can be used in a method of treating or prophylactically treating disorders in a human or non-human animal comprising administering to a human or non-human animal a therapeutically effective amount of a pharmaceutical composition according to the current invention.

The animal may be a land animal, an aquatic animal, an avian, or an amphibian. The animal may be a mammal, or a non-mammal. In a preferred embodiment, the animal is a human. In another embodiment, the animal is a non-human animal. The non-human animal can be an animal raised for human consumption or a domesticated animal. Examples of animals that can be administered the disclosed composition for use include, but are not limited to, ruminant species, such as a sheep, goat, cow, deer, bison, buffalo, elk, alpaca, camel or llama; ungulates, such as a horse, donkey, or pig; avians, such as chickens, including laying hens and broilers, turkey, goose, duck, Cornish game hen, quail, partridge, pheasant, guinea-fowl, ostrich, emu, swan, or pigeon; aquatic animals, such as an aquaculture species, such as fish (e.g., salmon, trout, tilapia, sea bream, carp, cod, halibut, snapper, herring, catfish, flounder, hake, smelt, anchovy, lingcod, moi, perch, orange roughy, bass, tuna, mahi mahi, mackerel, eel, barracuda, marlin, Atlantic ocean perch, Nile perch, Arctic char, haddock, hoki, Alaskan Pollock, turbot, freshwater drum, walleye, skate, sturgeon, Dover sole, common sole, wolfish, sablefish, American shad, John Dory, grouper, monkfish, pompano, lake whitefish, tilefish, wahoo, cusk, bowfin, kingklip, opah, mako shark, swordfish, cobia, croaker, or hybrids thereof, and the like), crustaceans (e.g., lobster, shrimp, prawns, crab, krill, crayfish, barnacles, copepods, and the like), or molluscs (e.g., squid, octopus, abalone, conchs, rock snails, whelk, clams, oysters, mussels, cockles, and the like). Additionally, or alternatively, the animal may be a companion animal, such as canines; felines; rabbits; rodents, such as a rat, mouse, hamster, gerbil, guinea pig or chinchilla; birds, such as parrots, canaries, parakeets, finches, cockatoos, macaws, parakeets or cockatiel; reptiles, such as snakes, lizards, tortoises or turtles; fish; crustaceans; and amphibians, such as frogs, toads and newts.

Accordingly, the invention also provides methods for the prevention and/or treatment of disorder; said method comprises administering to an individual (subject) in need thereof a pharmaceutical composition according to this invention. In some embodiments, said compound is administered orally or parenterally. In some embodiments, said compound is administered topically.

The compounds and compositions may also be used in in vitro methods, such as in vitro methods of administering a compound or composition to cells for screening purposes and/or for conducting quality control assays.

The compounds and compositions may also be used for ex-vivo treatment (e.g. Chimeric antigen receptor (CAR) T-cell therapy).

In certain embodiments, pharmaceutical compositions of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In some embodiments, the individual is an animal, preferably a mammal. In some embodiments, the individual is a primate, bovine, ovine, porcine, equine, canine, feline, or rodent. In some embodiments, the individual is a human. In some embodiments, the individual has any of tire diseases or disorders disclosed herein. In some embodiments, the individual is a risk of developing any of the diseases or disorders disclosed herein. In some embodiments, the individual is human. In some embodiments, the human is at least about or is about any of 21, 25, 30, 35, 40, 45, 50, 55, 60, 65, 70, 75, 80, or 85 years old. In some embodiments, the human is a child. In some embodiments, the human is less than about or about an 20, 18, 15, 12, 10, 8, 6, 5, 4, 3, 2, or 1 year.

### Articles of Manufacture and Kits

The present disclosure further provides articles of manufacture comprising a compound described herein or a salt thereof, a (pharmaceutical) composition described herein, or one or more-unit dosages described herein in suitable packaging. In certain embodiments, the article of manufacture is for use in any of the methods described herein. Suitable packaging is known in the art and includes, for example, vials, vessels, ampules, bottles, jars, flexible packaging, and the like. An article of manufacture may further be sterilized and/or sealed.

The present disclosure further provides kits for carrying out the methods of the invention, which comprises one or more compounds described herein or a (pharmaceutical) composition described herein. The kits may employ any of the compounds disclosed herein. In one embodiment, the kit employs a compound described herein or a salt thereof. The kits may be used for any one or more of the uses described herein. Kits generally comprise suitable packaging. The kits may comprise one or more containers comprising any compound described herein. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### Advantages and Applications

A big challenge in the field of mRNA therapeutics relies on the development of efficient delivery vectors that can (i) compact cargo such as mRNA, (ii) protect it from degradation, and (iii) ensure its intracellular delivery. Among all non-viral vectors, lipid nanoparticles (LNPs) are by far the most studied and advanced into clinical development.

These lipid nanoparticles are made of 4 components: an ionizable lipid, a helper lipid, a sterol and a PEGylated lipid. Over the last two decades, as the key component of this system, the ionizable lipid has been the subject of intense research. Structure-activity relationship is still in its infancy, but several parameters have been identified to deeply influence the polynucleotide encapsulation and release deeply (e.g., pKa, length of tail, shape, . . . ). Efforts have also been made to improve tolerability and biodegradability, leading to several lead compounds including the well-known DLin-MC3-DMA, SM-102 and ALC-0315 lipids. However, these systems remain perfectible, particularly in terms of biodistribution (off-target), (thermo) stability and reactogenicity; therefore, next-generation ionizable components for RNA delivery are still under development. The current invention brings an unexpected solution to the problem by providing a novel hybrid calixarene-lipid delivery system that overcome the disadvantages of conventional LNPs.

Calixarenes constitute a unique opportunity to design very modulable ionizable components for cargo delivery as the different phenolic units of calixarenes can be derivatized separately (particularly for calix[4] arenes), we can design molecules with a variable number of ionizable heads and hydrophobic tails. Several chemically different heads or tails can be combined to precisely control the encapsulation/delivery properties or to improve immunogenicity of the resulting delivery particles by introducing adjuvant patterns.

Furthermore, the inventors unexpectedly discovered that calixarenes in itself have an adjuvant effect when used in an LNP and that calixarenes can be used in an immunogenic composition, wherein said composition comprises an immunogenic component encapsulated in a lipid nanoparticle comprising said calixarene and wherein said LNP has an adjuvant effect in said immunogenic composition. Examples 4-8 describe this adjuvant effect.

In an embodiment, said lipid nanoparticle further comprising a PEGylated lipid. In a further embodiment, said lipid nanoparticle further comprises a sterol and/or a phospholipid. In an embodiment, said lipid nanoparticle comprises an ionizable calixarene and further comprises a PEGylated lipid, a sterol and a phospholipid. In an embodiment, said lipid nanoparticle comprises a cationic calixarene and further comprises a PEGylated lipid, a sterol, an ionizable lipid and a phospholipid.

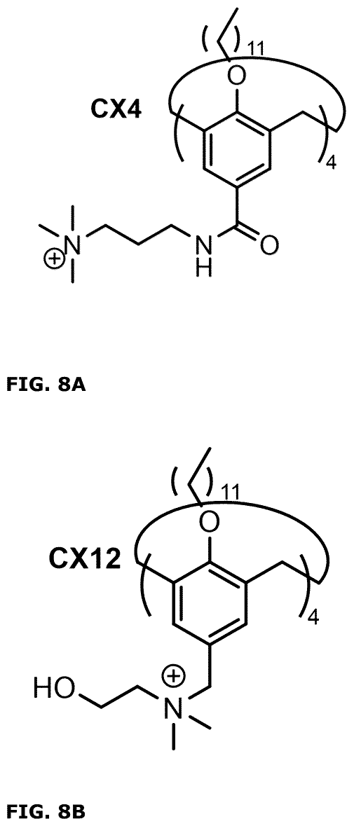

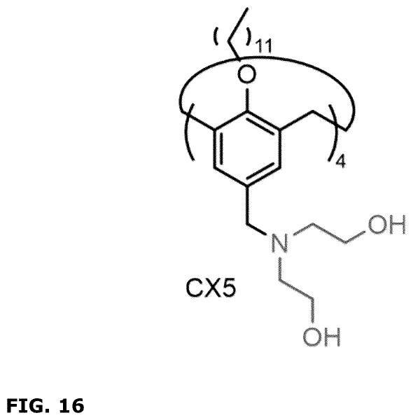

In a preferred embodiment, said lipid nanoparticle comprises at least one calixarene which is a cationic calixarene, comprising at least one moiety that is positively charged, and/or at least one calixarene which is an ionizable calixarene, comprising at least one moiety that is capable of associating with an ion and becoming positively charged. In a further embodiment, said positively charged moiety is an amine-bearing group comprising a secondary, ternary and/or quaternary amine. In an embodiment, said calixarene is an ionizable calix[4] arene having 4 head groups, wherein at least one of said head groups comprises at least one secondary or ternary amine (examples include for instance CX5 having 4 identical head groups having one ternary amine group, depicted in FIG. 16). In a further embodiment, said calixarene is a cationic calix[4] arene having 4 head groups, wherein at least one of said head groups comprises at least one quaternary amine. In an alternative further embodiment, said calixarene is a cationic calix[4] arene having 4 head groups, wherein at least one of said head groups comprises at least one quaternary amine (examples include for instance CX4 and CX12, each having 4 identical head groups having one quaternary amine group, depicted in FIG. 8).

When used in an immunogenic composition, said calixarene is preferably present in said lipid nanoparticle at a mass fraction of 0.1-60% (w/w). In an embodiment, when the calixarene is ionizable, it is present in said lipid nanoparticle at a mass fraction of 10-60% (w/w). In an embodiment, when used in an immunogenic composition, said ionizable calixarene is present in said lipid nanoparticle at a mass fraction of 10-60% (w/w), such as 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or 60% (w/w) or any value in between. In an embodiment, when used in an immunogenic composition, said ionizable calixarene is present in said lipid nanoparticle at a mass fraction between 10-15%, 15-20%, 20-25%, 25-30%, 30-35%, 35-40%, 40-45%, 45-50%, 50-55% or 55-60% (w/w). In an embodiment, when used in an immunogenic composition, said ionizable calixarene is present in said lipid nanoparticle at a mass fraction between 10-20%, 20-30%, 30-40%, 40-50% or 50-60% (w/w).

In an embodiment, when the calixarene is cationic, it is present in said lipid nanoparticle at a mass fraction of 0.1-50% (w/w). In an embodiment, when used in an immunogenic composition, said cationic calixarene is present in said lipid nanoparticle at a mass fraction of 0.1-50% (w/w), preferably 0.2-10% (w/w), such as 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5, 8%, 8.5%, 9%, 9.5%, 10% (w/w) or any value in between. In an embodiment, when used in an immunogenic composition, said cationic calixarene is present in said lipid nanoparticle at a mass fraction between 0.1-10%, 10-20%, 20-30%, 30-40% or 40-50% (w/w). In an embodiment, when used in an immunogenic composition, said cationic calixarene is present in said lipid nanoparticle at a mass fraction between 0.1-1%, 1-2%, 2-3%, 3-4%, 4-5%, 5-6%, 6-7%, 7-8%, 8-9%, 9-10%, 10-11%, 11-12%, 12-13%, 13-14% or 14-15% (w/w).

When used in said immunogenic composition, the calixarene mass comprised in said lipid nanoparticle is preferably less than 10 times, more preferably less than 5 times, more preferably lower than the nucleic acid mass encapsulated in said lipid nanoparticle.

As such, the calixarene is comprised in the delivery system of the current invention, providing the advantages as described above related to the efficient delivery (efficiently encapsulating nucleic acids, facilitating the encapsulation of very long RNA and/or minimizing the inherent toxicity of cationic components and/or reducing the non-specific adsorption of proteins that usually limits their efficacy), but also functioning on the level of immunogenic response in an immunogenic composition comprising an immunogenic component encapsulated in a lipid nanoparticle comprising said calixarene.

Similarly to the delivery system as described above, said lipid nanoparticle can further comprise an ionizable lipid. In an embodiment, said ionizable lipid is present in said immunogenic composition at a mass fraction of at most 60% (w/w).

When the calixarene is used in an immunogenic composition, said immunogenic component preferably comprises at least one nucleic acid molecule encoding at least one epitope of at least one antigen. In a preferred embodiment, said encoding nucleic acid molecule is a mRNA or an saRNA molecule.

Preferably, said immunogenic composition is intramuscularly administered to a subject.

In a further aspect, the disclosure relates to a vaccine, wherein said vaccine comprises an immunogenic component encapsulated in a lipid nanoparticle, wherein said lipid nanoparticle comprises at least one calixarene molecule and said LNP has an adjuvant effect in said vaccine.

In an embodiment, said vaccine is a DNA vaccine, said immunogenic component comprising DNA. In an embodiment, said vaccine is a RNA vaccine, said immunogenic component comprising RNA. In an embodiment, said vaccine is a cancer vaccine. In a preferred embodiment, said calixarene increases the immunogenicity of the immunogenic component after in vivo administration to a subject as measured by an elevated virus neutralizing titer (VNT) as compared to the same immunogenic composition not comprising a calixarene.

In an further aspect, the invention relates to a method of preparing an immunogenic preparation such as a vaccine, comprising a step of encapsulating an immunogenic component into a lipid nanoparticle, said lipid nanoparticle comprises at least one calixarene molecule and wherein said LNP has an adjuvant effect in said lipid nanoparticle.

As described above, said calixarene is preferably an ionizable or a cationic calixarene and said immunogenic component is preferably a nucleotide, such as a mRNA or an saRNA molecule.

As a comparison, the flexibility exhibited by traditional lipid or lipoid platform is by far lower. Traditional ionizable lipid usually bear only one ionizable head and introduction of supplementary hydrophobic tails to enhance their cone-shaped conformation usually requires multi-step synthesis, while this last is a natural property of calix[4] arenes. Concerning lipoids, their derivatization usually leads to symmetric compounds, without any possibility to modulate the nature of the ionizable heads and hydrophobic tails between them.

As recently demonstrated for several lipoid structures, increasing the number of ionizable head groups usually improves the encapsulation efficiency and the RNA release by increasing the N/P ratio (charge density) without modifying mass ratio between RNA and the ionizable component. With calix[4] arenes, this number of ionizable heads may easily reach 4, allowing us to consider the delivery of very long RNA molecules, such as saRNA, which remain very challenging with the current LNP technology based on ionizable lipids. In the same time, for traditional non-replicating mRNA, increasing the number of ionizable functions will allow us to reduce the quantity of ionizable component needed for good encapsulation and release, thereby diminishing the overall cost of the delivery system.

Further, the invention provides an alternative delivery system making use of cationic calixarene and ionizable lipids. Calixarenes are macrocyclic or cyclic oligomers based on methylene-bridged phenols, represented with the general structure below.

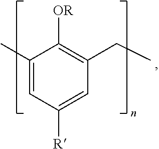

These compounds display several key features, which make them suitable for nucleic acid delivery when used in a delivery system similar to 5-component system, allowing controllable size and geometry. Compounds with a fixed number (4, 6, 8 . . . ) of phenolic units may be obtained. In addition, calix[4] arenes adopt a conical conformation easily. This conformation is a key property of traditional ionizable lipids that facilitates the RNA release through membrane destabilization and endosomal escape.

The encapsulation of negatively charged nucleotides largely depends on the interaction with positively charged compounds. The selection of such positively charged compounds is of major importance as they aid in the entrapment of RNA molecules and in facilitating endosomal escape.

As mentioned above, the calixarene-based delivery system of the current disclosure offers several advantages over conventional delivery systems. The delivery system can be tailored to specific applications by adjusting the composition of calixarene, phospholipid, sterol, PEGylated lipid and ionizable lipid.

The calixarene compounds have a controllable size and geometry. Compounds with a fixed number (4, 6, 8) of phenolic units may be obtained. In addition, the calixarene compounds adopt a conical conformation easily. This conformation is a key property to facilitate nucleotide (RNA) release through membrane destabilization and endosomal escape.

Furthermore, the calixarene compounds allow easy derivatization (on the lower and upper rim) to yield amphiphilic compounds that self-assemble into micelles in water, and can contain a payload, either in their cavity or through formation of ionic pairs between their functional groups and the charged payload (e.g., nucleotides).

In addition, the calixarene compounds allow to modulate the number of cationic head or ionizable head groups onto the same molecule. Increasing the number of cationic heads improves the encapsulation efficiency and the nucleotide (e.g. mRNA) release by increasing the N/P ratio without modifying mass ratio between mRNA and the cationic component.

Furthermore, calixarenes constitute a unique opportunity to design very modulable ionizable components for RNA delivery. Indeed, as the different phenolic units of calixarenes can be derivatized separately (particularly true for calix[4] arenes), molecules with a variable number of ionizable heads and hydrophobic tails can be designed. Several chemically different heads or tails could even be combined to precisely control the encapsulation/delivery properties or to improve immunogenicity of the resulting delivery particles by introducing adjuvant patterns. As a comparison, the flexibility exhibited by traditional lipid or lipoid platform is by far lower. Traditional ionizable lipids usually bear only one ionizable head and introduction of supplementary hydrophobic tails to enhance their cone-shaped conformation usually requires multi-step synthesis, while this last is a natural property of calixarenes. Concerning lipoids, their derivatization usually leads to symmetric compounds, without any possibility to modulate the nature of the ionizable heads and hydrophobic tails between them.

As recently demonstrated for several lipoid structures, increasing the number of ionizable head groups usually improves the encapsulation efficiency and the RNA release by increasing the N/P ratio (charge density) without modifying mass ratio between RNA and the ionizable component. With calixarenes, this number of ionizable heads may easily reach 8, allowing us to consider the delivery of very long RNA molecules, such as saRNA, which remain very challenging with the current LNP technology based on ionizable lipids. In the same time, for traditional non-replicating mRNA, increasing the number of ionizable functions will allow us to reduce the quantity of ionizable component needed for good encapsulation and release, thereby diminishing the overall cost of the delivery system.

In addition, the calixarene compounds allow to modulate the nature of the hydrophobic tail and to combine different hydrophobic motifs on the same molecule. These motifs can for instance provide adjuvanticity to the molecule or help with the endosomal escape.

The compounds according to the current invention allow for easy incorporation of biodegradable functionalities (ester, amide, disulfide bridge . . . ) to link the hydrophobic tails and the cationic heads on the macrocyclic core.

The present invention has broad applicability in various therapeutic and diagnostic settings, including gene therapy, RNA interference, genome editing, protein replacement therapy, drug delivery, and imaging. The improved cargo delivery system may enhance the efficacy and safety of these interventions, ultimately benefiting patients and advancing the field of medicine.

Although the present invention has been described in detail with reference to specific embodiments, it should be understood that various modifications and substitutions can be made without departing from the spirit and scope of the invention. Accordingly, the scope of the invention should not be limited to the specific embodiments described herein but should encompass all embodiments within the scope of the appended claims.

The invention is further described by the following non-limiting examples which further illustrate the invention, and are not intended to, nor should they be interpreted to, limit the scope of the invention.

### EXAMPLES

### Example 1: Example of a Possible Delivery System According to an Embodiment of The Current Invention: Use of Cationic Calixarene in a Delivery System

Molar ratios of components used for the formation of the delivery system:
Ionizable+ cationic components=25 to 40%
   Part of ionizable=80 to 95%
   Calixarene=1.25 to 8%
Helper lipid (phospholipids)=25 to 35%
Additional lipid (Sterol)=25 to 50%

The delivery systems are prepared according to the molar ratios using cationic calixarene. Specifically, DODAP is used as an ionizable component, Calixarene is used as a cationic component, DOPE is used as a phospholipid, Cholesterol is used as an additional lipid.

Key physico-chemical properties of the prepared delivery systems were assessed and the results are given below.
Particle Size=75-150 nm (PDI (polydispersity index)<0.3)
Encapsulation efficiency %>85%.
Protein expression is increased when calixarene is combined with DODAP

### Example 2: Development and Characterization of Lipid Nanoparticles According to an Embodiment of the Invention Comprising Ionizable Calix[4] Arenes

### INTRODUCTION

This example focuses on the design of ionizable calix[4] arenes and their use in RNA lipid nanoparticles (RNA-LNPs) to replace the traditional ionizable lipid. This modification is expected to change completely the behavior of the resulting delivery systems, especially their encapsulation and releasing capabilities. Calix[4] arenes display several key features that makes them suitable for nucleic acid delivery. The most important is their natural cone-shaped conformation that was found crucial for lipid nanoparticles/ionizable lipids to achieve high endosomal escape and favor the release of RNA in the cytosol. In addition, calix[4] arenes are platforms, which facilitates the synthesis of ionizable calix[4] arenes with 1, 2, 3, 4 or more amine heads, meaning that the charge density (number of amines/molecule) could be increased easily. This property helps the encapsulation of very long RNA, such as self-amplifying RNA (saRNA), by increasing the number of amines without changing the mass ratio between the ionizable component and RNA, while this task remains challenging with the current LNP technology not comprising calixarenes.

Ionizable Calix[4] Arenes Synthesis and Self-Assembly into Monodisperse Nanoparticles According to an Embodiment of the Invention Encapsulating mRNa-Fluc

A library of ionizable calixarenes was synthetized to better understand the structure-activity relationship. Calixarenes bearing one (CX14, CX16, CX24) or four (CX1, CX2, CX3, CX5, CX6, CX29) ionizable head groups (i-head) were synthesized (see FIG. 1). Ionizable heads were selected from the group of secondary amines (CX6, CX14) and ternary amines (CX1, CX2, CX3, CX5, CX16, CX24, CX29), cyclic or substituted with methyl or hydroxyethyl groups. Two biodegradable groups were also explored to link the ionizable head to the macrocyclic core and facilitate the metabolic degradation of the resulting compounds and avoid bioaccumulation (amide and ester links in CX2 and CX3/CX29 respectively).

All these calixarenes were self-assembled into nanoparticles according to an embodiment of the invention with a helper lipid (phospholipid), a sterol and a PEGylated lipid. Several helper (DOPE, DSPC) and PEG lipids (DMG-PEG2000, DSG-PEG2000) were explored. Ratios between these components were defined to produce stable monodisperse nanoparticles.

A minimum mass ratio of [Calixarenes+Lipids]/RNA is needed to obtain stable and monodisperse particles. This minimum ratio is ca. 20 for the 1-headed calixarene, while it may be decreased to 10 with the 4-headed calixarenes. This interesting result suggests that less material is needed to achieve similar encapsulation efficiency and monodispersity as compared to commonly used ionizable lipids (e.g., SM-102). The difference may be due to the increased charge density on the 4-headed ionizable calixarenes.

With respect to the helper lipid, the sterol and the PEGylated lipid, their mass fractions are conserved when moving from 1-headed to 4-headed systems. These lipid mass fractions (expressed in percent, wherein the mass of the delivery system without cargo represent 100%) are preferentially:
Ionizable calixarene: from 10 to 60%
Helper lipid: from 5 to 35%
Sterol: from 15 to 50%
PEGylated lipid: from 2 to 24%

It is worth noting that the PEGylated lipid is required to obtain stable monodisperse nanoparticles that do not aggregate as the PEGylated lipid ensures the shielding of particles and stabilizes their lipid-water interface. As a result, the PEGylate lipid mass fraction must be precisely controlled; a too low amount in PEG induces the aggregation of the particles, while a too high amount of PEGylated lipid limits their transfection capabilities (see in vivo protein expression results below). Examples of nanoparticles made with ionizable calixarenes according to an embodiment of the invention are given in Table 1 below.

TABLE 1
Examples of nanoparticles made with ionizable calixarenes and
encapsulating Fluc-mRNA (if not precised) or another RNA.

| | Helper | | PEGylated | | | |
|---|---|---|---|---|---|---|
| CX (mass | (mass | Sterol (mass | lipid (mass | (Lipids + CX)/RNA | Size (nm) | Encapsulation |
| ratio) | ratio) | ratio) | ratio) | mass ratio | [PDI] | efficiency (%) |
| CX1 | DOPE | Cholesterol | DMG- | 10 | 84 [0.08] | 97 |
| (45%) | (35%) | (15%) | PEG2000 | | | |
| | | | (5%) | | | |
| CX1 | DOPE | Cholesterol | DMG- | 10 | 70 [0.12] | 80 |
| (10%) | (35%) | (40%) | PEG2000 | | | |
| | | | (15%) | | | |
| CX1 | DOPE | Cholesterol | DMG- | 10 | 125 [0.10] | 96 |
| (60%) | (10%) | (25%) | PEG2000 | | | |
| | | | (5%) | | | |
| CX1 | DOPE | Cholesterol | DMG- | 30 | 56 [0.20] | 99 |
| (10%) | (25%) | (50%) | PEG2000 | | | |
| | | | (15%) | | | |
| CX1 | DOPE | Cholesterol | DMG- | 30 | 51 [0.08] | 99 |
| (60% | (10%) | (20%) | PEG2000 | | | |
| | | | (10%) | | | |
| CX1 | DOPE | Cholesterol | DMG- | 20 | 73 [0.04] | 99 |
| (36.5%) | (22.5%) | (36%) | PEG2000 | | | |
| | | | (5%) | | | |
| CX1 | DOPE | Cholesterol | DMG- | 19 | 53 [0.17] | 97 |
| (22%) | (29%) | (37%) | PEG2000 | | | |
| | | | (12%) | | | |
| CX2 | DOPE | Cholesterol | DMG- | 20 | 54 [0.15] | 98 |
| (24%) | (28%) | (36%) | PEG2000 | | | |
| | | | (11%) | | | |
| CX3 | DOPE | Cholesterol | DMG- | 20 | 56 [0.14] | 98 |
| (26%) | (27%) | (35%) | PEG2000 | | | |
| | | | (11%) | | | |
| CX5 | DOPE | Cholesterol | DMG- | 20 | 55 [0.19] | 97 |
| (24%) | (28%) | (36%) | PEG2000 | | | |
| | | | (11%) | | | |
| CX5 | DSPC | Cholesterol | DMG- | 19 | 67 [0.11] | 90 |
| (25%) | (19%) | (44%) | PEG2000 | | | |
| | | | (12%) | | | |
| CX5 | DOPE | Cholesterol | DMG- | 32 | 47 [0.13] | 98 |
| (23%) | (11%) | (43%) | PEG2000 | | | |
| | | | (23%) | | | |
| CX5 | DOPE | Cholesterol | DMG- | 19 | 47 [0.13] | 98 |
| (25%) | (12%) | (46%) | PEG2000 | | | |
| | | | (18%) | | | |
| CX5 | DOPE | Cholesterol | DMG- | 25 | 63 [0.04] | 99¹ |
| (28%) | (27%) | (34%) | PEG2000 | | | |
| | | | (11%) | | | |
| CX5 | DOPE | Cholesterol | DMG- | 25 | 63 [0.07] | 98² |
| (28%) | (27%) | (34%) | PEG2000 | | | |
| | | | (11%) | | | |
| CX5 | DOPE | Cholesterol | DMG- | 39 | 65 [0.15] | 91³ |
| (28%) | (27%) | (34%) | PEG2000 | | | |
| | | | (11%) | | | |
| CX16 | DOPE | Cholesterol | DMG- | 29 | 113 [0.05] | 96 |
| (37%) | (28%) | (31%) | PEG2000 | | | |
| | | | (4%) | | | |
| CX16 | DOPE | Cholesterol | DMG- | 22 | 61 [0.13] | 99 |
| (49%) | (24%) | (22%) | PEG2000 | | | |
| | | | (5%) | | | |
| CX16 | DSPC | Cholesterol | DMG- | 20 | 81 [0.10] | 98 |
| (54%) | (6%) | (36%) | PEG2000 | | | |
| | | | (4%) | | | |
| CX16 | DSPC | Cholesterol | DMG- | 37 | 58 [0.18] | 99 |
| (29%) | (31%) | (36%) | PEG2000 | | | |
| | | | (4%) | | | |
| CX29 | DOPE | Cholesterol | DMG- | 20 | 51 [0.11] | 98 |
| (27%) | (27%) | (35%) | PEG2000 | | | |
| | | | (11%) | | | |
| CX29 | DOPE | Cholesterol | DMG- | 20 | 98 [0.20] | 92 |
| (69%) | (7%) | (17%) | PEG2000 | | | |
| | | | (7%) | | | |

¹mRNA encoding for the spike protein from SARS-CoV2
²mRNA encoding for the glycoprotein G from Rabies virus
³saRNA encoding for FLuc

In Vitro Transfection of mRNA-FLuc Using Delivery Systems According to an Embodiment of the Invention

In vitro potency assays in Jurkat cells using Fluc mRNA as a reporter gene were performed. Average luminescence was measured 24 h after transfection of 100 ng mRNA in Jurkat cells. Cells were seeded at day 1 (10000 cells/well in a 96-well plate) and cells were transfected at day 2. The luminescence was read at day 3 after luciferin addition. The first results (Table 2) demonstrated that CX compounds with ternary amines are more potent. At the same time, these assays provided evidence of the superiority of the 4-headed calixarenes over the 1-headed calixarenes. For the specific group of 4-headed calixarenes, we further demonstrated that increasing the number of hydroxyethyl functions improves the potency.

TABLE 2
Average luminescence measured 24 h after transfection of 100 ng mRNA in Jurkat
cells using a delivery system according to an embodiment of the invention.

| | | | PEGylated | (Lipids + | Average |
|---|---|---|---|---|---|
| CX | Helper | Sterol | lipid | CX)/RNA | luminescence |
| (mass ratio) | (mass ratio) | (mass ratio) | (mass ratio) | mass ratio | (a.u.) |
| CX1 | DOPE | Cholesterol | DMG-PEG2000 | 19 | 21196 |
| (22%) | (29%) | (37%) | (12%) | | |
| CX2 | DOPE | Cholesterol | DMG-PEG2000 | 20 | 723 |
| (24%) | (28%) | (36%) | (11%) | | |
| CX3 | DOPE | Cholesterol | DMG-PEG2000 | 20 | 2324 |
| (26%) | (27%) | (35%) | (11%) | | |
| CX5 | DOPE | Cholesterol | DMG-PEG2000 | 20 | 21368 |
| (24%) | (28%) | (36%) | (11%) | | |
| CX14 | DOPE | Cholesterol | DMG-PEG2000 | 27 | 104 |
| (37%) | (21%) | (37%) | (5%) | | |
| CX16 | DOPE | Cholesterol | DMG-PEG2000 | 29 | 754 |
| (37%) | (28%) | (31%) | (4%) | | |
| CX24 | DOPE | Cholesterol | DMG-PEG2000 | 30 | 205 |
| (38%) | (28%) | (31%) | (4%) | | |
| CX29 | DOPE | Cholesterol | DMG-PEG2000 | 20 | 1492 |
| (27%) | (27%) | (35%) | (11%) | | |

CX: calixarene
a.u.: arbitrary unit

mRNA-FLuc In Vivo Protein Expression is Modulated by the Mass Ratios

Within the range of optimal mass ratios defined above to obtain stable monodisperse particles, an experiment was designed to further refine these ratios and maximize the protein expression in vivo. The best in vitro performer (CX5) was selected, and nanoparticles, where the total (lipid+CX)/RNA mass ratio was varied between 15 and 45, the helper content between 10% and 30%, and the PEGylated lipid between 10% and 24%, were tested.

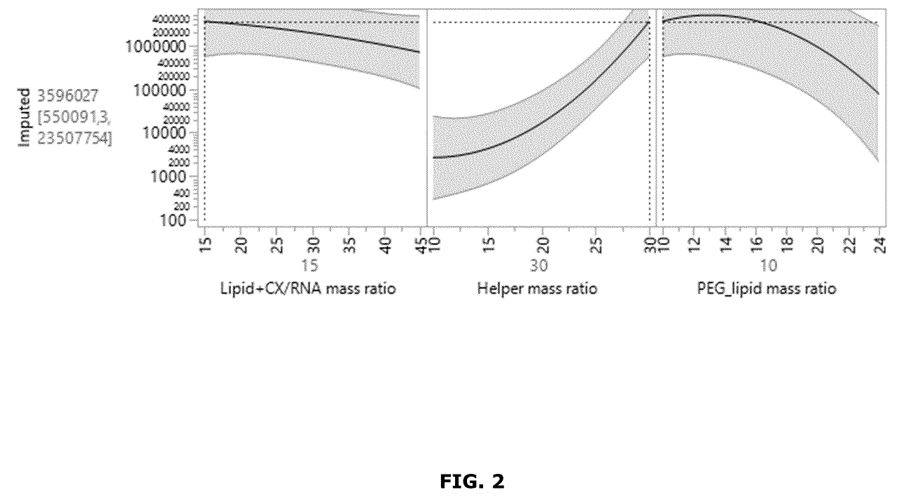

1 μg of Fluc mRNA was administrated via an intramuscular route and the luminescence at the injection site was used to estimate the effect of the above-mentioned factors. Visualization of the estimated model (average+95% confidence intervals) is given in FIG. 2 and clearly indicates favorable helper and PEG lipid mass ratios to increase the in vivo protein expression. As mentioned above, a minimal PEG amount is preferred, but a lower limit is needed to achieve colloidal stability. FIG. 2 shows visualization of the estimated model (average+95% confidence interval) of in vivo luminescence as a function of the (lipid+CX)/RNA mass ratio, the helper mass ratio and the PEG lipid mass ratio.

After having optimized the molar ratios using CX5 in vivo, different calixarenes selected from those depicted in FIG. 1 were compared. 4-headed CX1, CX2, CX3, CX5 were formulated at the optimal ratio defined for CX5. The 1-headed CX16 was also included in the study at its own optimal ratios defined in vitro. Results of protein expression per calixarene delivery system (geometric mean signal with 95% confidence intervals) at 3 h, 6 h and 9 h after the intramuscular injection of 1 μg of Fluc mRNA are shown in FIG. 3. 1 μg of RNA was administrated to both left and right hindlimb of mice. A DoE was used to randomize samples and avoid interactions between delivery systems and luminescence was estimated by integrating the radiance at the injection site. These data showed that CX5, the 4-headed calixarene bearing the most hydroxyethyl groups, performs best. Overall, this experiment indicated that the kinetic of protein expression is different between 1-headed and 4-headed calixarenes. While the signal of CX16 (1-headed) rapidly decreased, the protein expression induced by the 4-headed calixarenes (CX1, CX2, CX3 and CX5) remained stable over the different timepoints (from 3 to 9 h after injection). This result shows again the superiority of the 4-headed calixarenes over the 1-headed for achieving a stable and high protein expression. This observation is strengthened by the lower amount of 4-headed calixarenes needed to reach this performance (ca. 5 μg 4-headed CX/μg RNA vs. ca. 10 μg 1-headed CX/μgRNA).

### saRNA-FLuc Encapsulation in Nanoparticles According to an Embodiment of the Invention

While keeping the optimized mass ratio between the four components (CX, helper, cholesterol, PEG lipid), saRNA encoding for FLuc was then encapsulated using CX5 by varying the CX/saRNA mass ratio. Over the whole screening space (CX/saRNA mass ratio from 2.3 to 9.2), stable nanoparticles with high encapsulation efficiency (>80%) were obtained (see FIG. 4). FIG. 4 shows the encapsulation efficiency and in vitro protein expression as a function of the CX5/saRNA mass ratio of various delivery systems according to an embodiment of the invention. Two independent samples were produced per condition. Interestingly, as shown in FIG. 4, the encapsulation efficiency increased when the CX/saRNA mass ratio was increased from 2.3 to 5.8, but then remained stable at a mass ratio of 9.2, showing a saturation effect of the CX quantity needed to reach a maximal encapsulation efficiency. However, further increasing the amount of CX5 allowed us to increase the protein expression. It is worth mentioning that this optimized mass ratio (9.2) remains small in comparison to those needed (ca. 20 μg i-lipid/μg RNA) to reach similar properties with saRNA (encapsulation efficiency and in vitro protein expression) with well-known ionizable lipids used in the field (SM-102, ALC-0315).

### In Vivo saRNA-Fluc Delivery Efficiency of a Delivery System According to an Embodiment of the Current Invention Compared to a Reference Delivery System Without a Calixarene

The great performance demonstrated in vitro was also confirmed in vivo. The intramuscular injection of 0.2 μg saRNA encoding for FLuc induced a strong production of luciferase that increased over time until at least 1 week (FIG. 5). Similar performance was obtained with a reference system (SM-102 based LNP) but with approximatively a two-fold higher quantity in ionizable component (20.9 vs. 9.2), showing again the superiority of using 4-headed calixarenes with high charge density. FIG. 5 shows the geometric mean signal (+signal from each mouse) per delivery system (delivery system comprising CX5 in blue, reference delivery system in red) over time. 0.2 μg of saRNA was administrated to the right hindlimb of each mice. Luminescence was estimated by integrating the radiance at the injection site.

Pre-Clinical Evaluation of a Delivery System According to an Embodiment of the Invention Comprising mRNA Encoding for the G-Protein of the Rabies Virus

A delivery system comprising CX5 was tested in an immunogenicity study using the glycoprotein G from Rabies as a model antigen. Mice were vaccinated following a 21 days prime-boost regimen using 0.6 μg or 2.5 μg of mRNA per dose. As shown in FIG. 6, CX5 yielded strong VNTs (virus neutralization titers), well above the correlate of protection (0.5 IU/mL), using the two dosing regimens (0.6 or 2.5 μg), without inducing any adverse effect (loss of weight, impact on spleen, liver and kidneys weight, inflammation or an excessive reactogenic response) on the animals. FIG. 6 shows VNTs serum levels measured after 15 (after prime), 35 and 65 (after boost) days (left: 0.6 μg doses, right: 2.5 μg doses).

### Cryo-TEM Imaging of Nanoparticles Made with Ionizable Calix[4] Arenes According to An Embodiment of the Invention

CX5: DOPE: cholesterol:DMG-PEG2000 nanoparticles encapsulating mRNA-FLuc were imaged using cryo-TEM (Error! Reference source not found.). Lacey Formvar/Si monoxide grids, 300 mesh Cu (Ted Pella Inc, 01887-F) were glow discharged using an ELMO glow discharger for 20 s at 4.5 mA. 3.5 μl of sample was applied to the grid plunged in liquid ethane at 95% humidity and 20 degrees Celsius in the chamber on a Gatan Cp-3 using 4s double sided blotting. The cryo grids were imaged using a JEOL1400 microscope equipped with a TVIPS F416 at 60 000× and Gatan 626 side entry holder at 60 k magnification. 60 k Magnification images are 4k×4k, with a pixel size of 0.194 nm/px. Sample was applied undiluted. LNPs are monodisperse, with the smallest and largest LNPs around 40 nm and 80 nm respectively, which is in good agreement with DLS measurements (Z-average of ca. 50 nm).

### Example 3: Development and Characterization of Lipid Nanoparticles According to an Embodiment of the Invention Comprising Cationic Calix[4] Arenes

### INTRODUCTION

This example focuses on cationic calixarenes and their incorporation as a fifth component into lipid nanoparticles (LNPs) made of an ionizable lipid, a helper lipid, a sterol, and a PEG lipid.

By adding cationic calixarenes the behaviour of the resulting delivery systems is completely changed, especially their encapsulation and releasing capabilities. As described above, calix[4] arenes actually display several key features that makes them suitable for nucleic acid delivery. The most important is their natural cone-shaped conformation that was deemed crucial for lipid nanoparticles/ionizable lipids to achieve high endosomal escape and favor the release of RNA in the cytosol. In addition, as a platform, the calix[4] arenes facilitate the access to cationic compounds with 1, 2, 3, 4 or more quaternary amine heads, meaning that the charge density (number of amines/molecule) could be increased easily. This property should help us to consider the encapsulation of very long RNA, such as self-amplifying RNA (saRNA) by adding a small amount of cationic calixarene into an LNP, while this task remains challenging with the current LNP technology. Furthermore, incorporation of the cationic calixarene into an LNP allows (i) to minimize the inherent toxicity of these cationic components by combining them with non-toxic biocompatible lipids, and (ii) to reduce the non-specific adsorption of proteins that usually limits their efficacy.

Cationic calix[4] arenes synthesis and addition into monodisperse lipid nanoparticles according to an embodiment of the invention encapsulating mRNA-FLuc Two cationic calixarenes were synthesized and tested, CX4 and CX12 (see FIG. 8). Experiments demonstrated that these cationic calixarenes can be embedded into lipid nanoparticles encapsulating RNA (Table 3) and made with:
Ionizable lipids: DODAP, DLin-DMA, DLin-MC3-DMA, ALC-0315, SM-102
Helper lipids: DOPE, DOPC
Sterol: cholesterol
PEG lipids: DMG-PEG2000, DSG-PEG2000

TABLE 3
Examples of nanoparticles made with cationic calixarenes and
encapsulating an Fluc-mRNA (if not precised) or another RNA.

| | | | PEGylated | | | | |
|---|---|---|---|---|---|---|---|
| i-lipid | Helper | Sterol | lipid | CX | | Size | |
| (mass | (mass | (mass | (mass | (mass | (Lipids + CX)/RNA | (nm) | Encapsulation |
| fraction) | fraction) | fraction) | fraction) | fraction) | (mass ratio) | [PDI] | efficiency (%) |
| DODAP | DOPE | Cholesterol | DMG- | CX12 | 25.7 | 130 | 98 |
| (42.4%) | (31.2%) | (22.9%) | PEG2000 | (1.4%) | | [0.12] | |
| | | | (2.1%) | | | | |
| DODAP | DOPE | Cholesterol | DMG- | CX12 | 24.4 | 158 | 96 |
| (39.9%) | (23.0%) | (30.3%) | PEG2000 | (4.5%) | | [0.15] | |
| | | | (2.3%) | | | | |
| DLin- | DOPE | Cholesterol | DSG- | CX4 | 27.1 | 143 | 97 |
| DMA | (16.6%) | (37.2%) | PEG2000 | (0.8%) | | [0.19] | |
| (39.1%) | | | (6.3%) | | | | |
| DLin- | DOPE | Cholesterol | DSG- | CX12 | 26.7 | 163 | 98 |
| DMA | (16.9%) | (38.1%) | PEG2000 | (0.7%) | | [0.13] | |
| (39.8%) | | | (4.5%) | | | | |
| DLin- | DOPE | Cholesterol | DMG- | CX12 | 26.6 | 160 | 97 |
| DMA | (16.9%) | (38.3%) | PEG2000 | (0.7%) | | [0.17] | |
| (39.8%) | | | (4.3%) | | | | |
| DLin- | DOPE | Cholesterol | DMG- | CX4 | 26.8 | 154 | 98 |
| DMA | (16.9%) | (37.9%) | PEG2000 | (0.8%) | | [0.19] | |
| (39.7%) | | | (4.7%) | | | | |
| DLin- | DOPE | Cholesterol | DMG- | CX4 | 27.0 | 154 | 98¹ |
| DMA | (16.7%) | (37.5%) | PEG2000 | (0.8%) | | [0.25] | |
| (39.3%) | | | (5.6%) | | | | |
| DLin- | DOPE | Cholesterol | DMG- | CX12 | 26.7 | 84 | 71² |
| DMA | (16.9%) | (38.0%) | PEG2000 | (0.7%) | | [0.21] | |
| (39.7%) | | | (4.7%) | | | | |
| DLin- | DOPE | Cholesterol | DMG- | CX12 | 26.7 | 105 | 77³ |
| DMA | (16.9%) | (38.0%) | PEG2000 | (0.7%) | | [0.11] | |
| (39.7%) | | | (4.7%) | | | | |
| DLin- | DOPE | Cholesterol | DMG- | CX12 | 27.4 | 123 | 96 |
| MC3- | (16.5%) | (36.9%) | PEG2000 | (0.7%) | | [0.12] | |
| DMA | | | (5.6%) | | | | |
| (40.3%) | | | | | | | |
| SM-102 | DOPE | Cholesterol | DMG- | CX12 | 28.6 | 82 | 97 |
| (42.8%) | (15.8%) | (35.4%) | PEG2000 | (0.7%) | | [0.16] | |
| | | | (5.3%) | | | | |
| ALC- | DOPE | Cholesterol | DMG- | CX12 | 29.3 | 139 | 93 |
| 0315 | (15.4%) | (34.7%) | PEG2000 | (0.6%) | | [0.07] | |
| (45.0%) | | | (4.3%) | | | | |

¹mRNA encoding for the glycoprotein G from Rabies virus
²mRNA encoding for the spike protein from SARS-CoV2
³saRNA encoding for the spike protein from SARS-CoV2

LNPs are Stable with a Large Fraction of Cationic Calixarene

A design of experiment (DoE) was used to model the effect of CX12 mass per LNP (expressed as milligrams of CX12 per milligram of RNA dose) on physicochemical properties. The other lipids (DLin-DMA, DOPE, cholesterol, DMG-PEG) and mRNA (Firefly luciferase) masses were maintained constant. Results of the experiment and of the modeling are shown in table 4 and FIG. 9. LNPs made with between 0 and 20.7 times more CX12 than mRNA were all found to be monodisperse and encapsulate more than 95% of the RNA. The addition of CX12 was found to increase both size (from 77 to 129 nm) and charge (from 5.8 to 11.0 mV).

TABLE 4
Design of experiment used to model the effect of CX12 mass per
LNP on physicochemical properties. Results of the modeling.

| CX12 | Z- | | RNA | | ζ- |
|---|---|---|---|---|---|
| mass per | average | | conc. | EE | potential |
| mg RNA | (nm) | PDI | (ng/μL) | (%) | (mV) |
| 1.7 | 83 | 0.09 | 52 | 97.6 | 4.82 |
| 0 | 87 | 0.11 | 73 | 97.2 | 3.41 |
| 20.7 | 245 | 0.18 | 73 | 98.9 | 17.53 |
| 0 | 86 | 0.08 | 65 | 96.8 | 2.33 |
| 20.7 | 79 | 0.15 | 60 | 98.2 | 4.51 |
| 20.7 | 78 | 0.09 | 57 | 98.1 | 7.18 |
| 1.7 | 85 | 0.17 | 58 | 97.9 | 7.04 |
| 6.2 | 76 | 0.1 | 69 | 99.5 | 11.04 |
| 1.7 | 72 | 0.05 | 59 | 97.9 | 7.02 |
| 0 | 93 | 0.21 | 66 | 96.8 | 2.46 |
| 6.2 | 78 | 0.12 | 66 | 99.5 | 11.5 |
| 6.2 | 77 | 0.1 | 80 | 99.6 | 11.98 |

### Pre-Clinical Evaluation of Candidate LNPs According to an Embodiment of the Invention Comprising a Cationic Calixarene and DLin-DMA

4 different LNPs were fabricated using combinations of CX4 and CX12, and DMG-PEG2000 and DSG-PEG2000. In all cases, DLin-DMA was used as the ionizable lipid. mRNA encoding for G-protein of the Rabies virus was used. Physicochemical characteristics of the LNPs produced are shown in Table 5.

TABLE 5
Physicochemical characteristics of 4 LNPs according to an embodiment
of the invention comprising a cationic calixarene

| | | Zeta- | Z- | | |
|---|---|---|---|---|---|
| | Mass fraction | Potential | Average | | EE |
| LNP | (%) | (mV) | (nm) | PDI | (%) |
| DLin-DMA:DOPE:cholesterol:DMG- | 39.3:16.7:37.5:5.6:0.8 | 1.9 | 195.7 | 0.36 | 98.0 |
| PEG2000:CX4 | | | | | |
| DLin-DMA:DOPE:cholesterol:DSG- | 39.3:16.7:37.5:5.9:0.7 | 2.8 | 153.9 | 0.25 | 97.6 |
| PEG2000:CX4 | | | | | |
| DLin-DMA:DOPE:cholesterol:DMG- | 39.4:16.7:37.6:5.6:0.7 | 1.1 | 165.4 | 0.38 | 97.6 |
| PEG2000:CX12 | | | | | |
| DLin-DMA:DOPE:cholesterol:DSG- | 39.2:16.7:37.4:5.9:0.8 | 1.3 | 220.2 | 0.25 | 98.6 |
| PEG2000:CX12 | | | | | |

BALB/c mice were vaccinated following a prime-boost regimen 21 days apart (prime at day zero and boost at 21 days) with DLin-DMA: CX4 or DLin-DMA: CX12 LNPs with two different PEGylated lipids. VNTs serum levels were measured after 15 or 35 days for 0.6 or 2.5 μg RNA doses. LNPs were frozen at −80° C. for shipment and storage. The same batches were used to prime and boost the mice. Geometric mean titers are shown in FIG. 10. VNTs produced using all LNPs yield strong VNTs after 35 days. The raw data shows a dose response effect, with doses of 2.5 μg on average higher than the 0.6 μg after both 15 and 35 days. For all LNPs and doses, the VNTs are well above the correlate of protection (shown as the dashed black line at 0.5 a.u.). These results demonstrate that cationic calixarenes combined with DLin-DMA as the ionizable lipid yields potent LNPs. These results also suggest that the PEGylated lipid choice impacts the immune response: a difference as small as 4 additional carbons on the two alkyl chains of the PEGylated lipid can significantly decrease the immune potency of an otherwise identical LNP.

saRNA Encapsulation in an LNP According to an Embodiment of the Invention Comprising DLin-DMA and CX12

A DLin-DMA: DOPE: cholesterol:DMG-PEG2000: CX12 LNP was used to encapsulate saRNA encoding for the full-length spike protein of SARS-COV-2. The resulting LNPs was found monodisperse with a Z-average of 120 nm (see FIG. 11A) and encapsulated 76.6% of the saRNA. This result demonstrates that this formulation can be used for much larger RNA such as saRNA. This result is particularly interesting as common ionizable lipids are difficult to use to encapsulate saRNA. This was demonstrated with an saRNA of similar length and encoding for an undisclosed gene of interest. The saRNA was formulated using a SM-102: DSPC: cholesterol: DMG-PEG2000 (50:10:38.5:1.5 molar ratio). The resulting LNP was found polydisperse (see FIG. 11B).

### Example 4: LNPs Comprising Cationic Calix[4] Arenes Act as Adjuvants Using the Pre-Fusion Stabilized Spike Protein of SARS-COV-2

Experiments were performed using ionizable lipids (i-lipid) to fabricate LNPs along with a helper, cholesterol, a PEGylated lipid and mRNA. The inventors unsurprisingly found that LNPs made with DODAP (1,2-dioleoyl-3-dimethylammonium propane) yielded poor cytosolic delivery and encapsulation efficiency. Yet, the inventors found that this could be ameliorated by adding a cationic lipid to the LNP and by carefully selecting the helper lipid (a phospholipid such as DOPE, DOPC and DSPC), the molar ratios between lipids and the amine to phosphate (N: P) ratio. The cationic lipids tested included DOTAP, DDAB and DC-cholesterol. The inventors also tested a new class of molecule for integration in LNPs, namely cationic calix[4] arenes.

After optimizing the fabrication process and molar composition, two LNPs were used to encapsulate mRNA encoding for Firefly Luciferase. 1005-034-08 included DOTAP and I005-203-401 included the cationic calixarene CX12 (FIG. 8B) as the cationic component, while both used DODAP as the i-lipid. The LNPs were formulated in a TRIS 20 mM sucrose 8% w/v pH 7.4 buffer. Both LNPs encapsulated more than 95% of the mRNA and were monodisperse (see Table 6 for physicochemical characterization).

TABLE 6
Physicochemical characteristics of LNPs used in the IVIS study.

| | ζ-potential | | | EE | | Cationic |
|---|---|---|---|---|---|---|
| LNP ID | (mV) | Z_average_(nm) | PDI | (%) | i-lipid | component |
| I005-034-08 | −1.99 | 89.0 | 0.225 | 95 | DODAP | DOTAP |
| I005-203-401 | −3.02 | 129.7 | 0.123 | 98 | DODAP | CX12 |

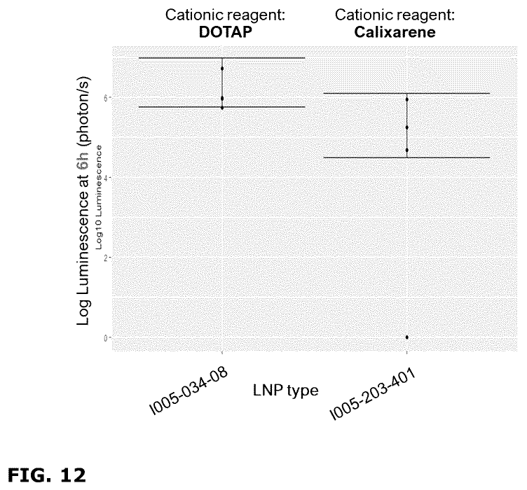

1 μg RNA encapsulated in each LNP type was administrated intramuscularly (IM) in BALB/c mice. Luciferin was administrated 6 hours, 12 hours and 24 hours after LNPs injection, and the luminescence was measured in an in vivo imaging system (IVIS). The radiance was integrated over the region of interest (ROI) to estimate the luciferase expression obtained with these two systems. Results are shown in FIG. 12 for the 6 hours timepoint. Both LNPs yielded similar protein expression.

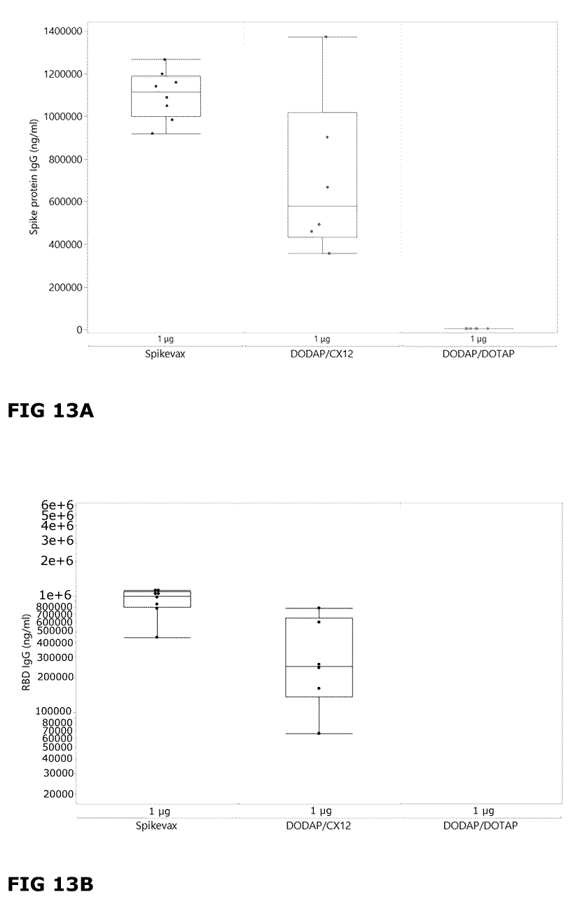

Using a SARS-COV-2 model, we discovered an unexpected adjuvant effect for LNPs comprising cationic calixarenes (here CX12). A nanoparticle combining a well-known poorly efficient ionizable lipid DODAP and CX12 was demonstrated to elicit a potent immune response. After a 21 days prime-boost regimen using 1 μg of RNA per dose, the measured antibody titers (anti-spike & anti-RBD) were estimated at a slightly lower level, but not statistically different as compared to the Spikevax vaccine from Moderna (Spike-encoded RNA encapsulated in SM-102 LNPs) (see FIGS. 13A and 13B).

It is worth noting that the protein expression induced by the DODAP/CX12 system was previously evaluated 3 log below the SM-102 LNP comparator, showing the potent adjuvant effect of CX12. This effect is clearly attributed to the calixarene as another DODAP-based system supplemented with another cationic agent (DOTAP) did not exhibit any immunogenic effect, while inducing a slightly higher protein expression in mice. This result is particularly attractive as the needed amount of CX12 is extremely low (~0.35 μg/μg of RNA) to achieve such a performance.

### Example 5: Extension to Other Antigens-Potent LNPs for a Rabies Vaccine when a Cationic Calixarene was Used According to an Embodiment of the Invention

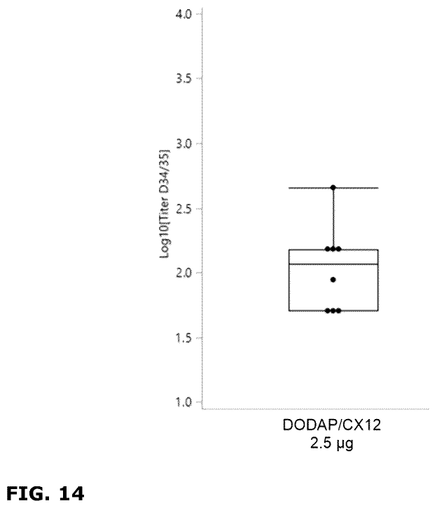

To further demonstrate the adjuvanticity of the LNPs comprising the cationic calixarene CX12, a different pre-clinical model was used. The same LNP was used to encapsulate mRNA encoding for the G-protein of the Rabies virus. 2.5 μg mRNA was administrated in mice hind legs and a 21 days prime-boost regimen was followed. Virus neutralizing titers (VNTs) were measured in blood serum 35 days after the prime. Results are presented in FIG. 14 and show neutralizing titers (VNTs) well above the correlate of protection (0.5 IU/mL). These results demonstrate that an LNP comprising a cationic calixarene such as CX12 can be used to adjuvant and mount an immune response against different antigens.

These results are particularly attractive as the amount of cationic calixarene needed is extremely low (~0.35 μg/μg of RNA) to achieve such a performance. By comparison, when cationic calixarene are used as a DNA transfection agent in the literature, the amount of calixarene needed was reported to be at least 25-fold higher (>10 μg/μg of DNA). This demonstrates the synergy between the cationic calixarene and the LNP architecture.

### Example 6: Extension to Other LNPs and Other Cationic Calix[4] Arenes for Use According to an Embodiment of the Invention

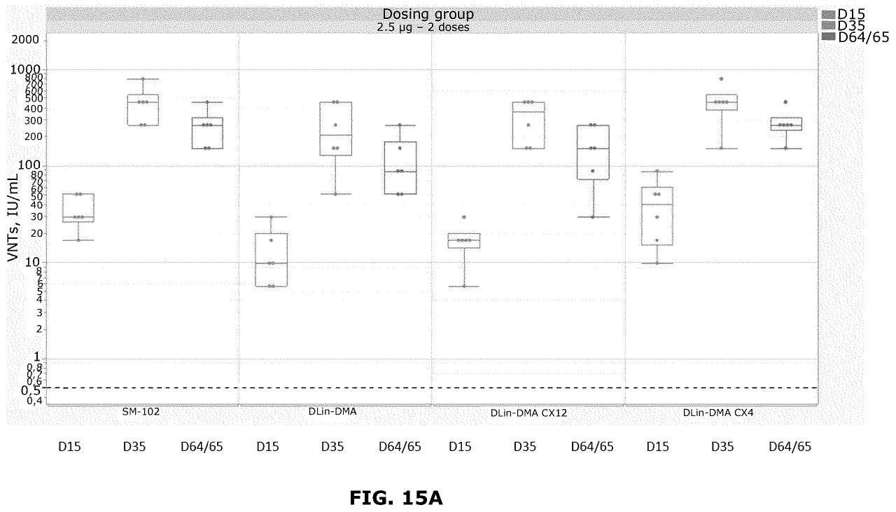

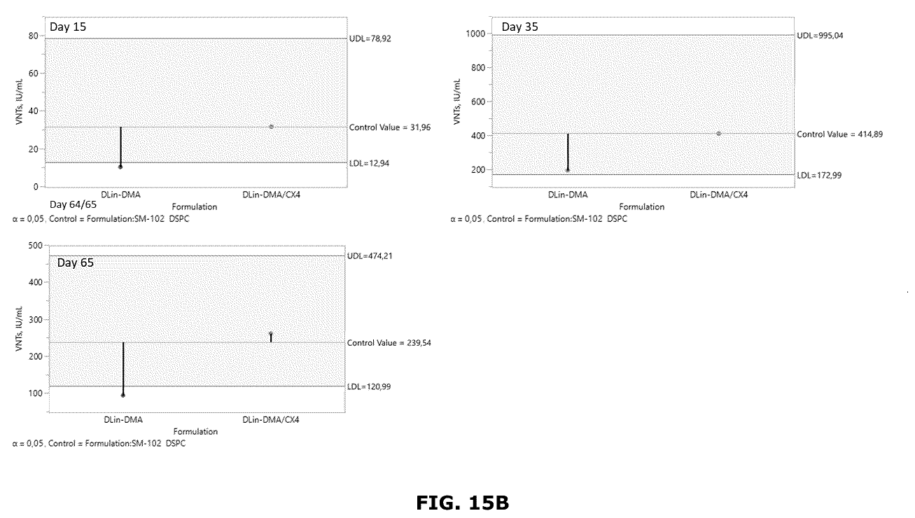

The use of cationic calix[4] arenes can be extended to other LNPs. LNPs made with N,N-dimethyl-2,3-bis[(9Z,12Z)-9,12-octadecadien-1-yloxy]-1-propanamine (DLin-DMA), a helper, cholesterol and a PEGylated lipid were fabricated with or without integrating a cationic calix[4] arenes. Two different calix[4] arenes were tested, namely CX12 and CX4 (see FIG. 8). All LNPs were found monodisperse and encapsulated mRNA (see Table 7). All LNPs encapsulated mRNA encoding for the G-protein of the Rabies virus. 0.6 or 2.5 μg mRNA was administrated in mice hind leg and a 21 days prime-boost regimen was followed. Virus neutralizing titers (VNTs) were measured in blood serum after 15, 35 and 64/65 days. Results are shown in FIG. 15A and FIG. 15B.

TABLE 7
Physicochemical characteristics of LNPs fabricated

| | Zeta- | Z- | | |
|---|---|---|---|---|
| LNP | Potential (mV) | Average (nm) | PDI | EE (%) |
| DLin-DMA | 2.1 | 201.1 | 0.12 | 97.9 |
| DLin-DMA: CX12 | 1.1 | 165.4 | 0.38 | 97.6 |
| DLin-DMA: CX4 | 1.9 | 195.7 | 0.36 | 98.0 |

As shown in FIG. 15A, VNTs obtained using DLin-DMA LNPs boosted with either CX4 or CX12 are well above the correlate of protection (0.5 IU/mL).

FIG. 15B shows the virus neutralizing titers (VTNs) in serum measured after 15 (after prime), 35 and 65 days (after boost) days in mice which were vaccinated using SM-102 LNPs (control), DLin-DMA LNPs and DLin-DMA/CX4 LNPs. A 21 days prime-boost regimen using 2.5 μg of mRNA per dose was used (RNA encoding for the glycoprotein G from the Rabies virus). The results are shown as a comparison with the control delivery system (SM-102 LNPs). UDL=upper decision limit, LDL=lower decision limit. All points crossing the limits indicate a significant difference as compared to the control.

This result shows again the potent adjuvant and synergetic effect of adding a cationic calixarene into a lipid nanoparticle. It also demonstrates that the adjuvanticity is not limited to a specific cationic calix[4] arenes, as shown by the potent responses obtained with CX4.

### Example 7: a Large Range of Calixarene Fractions Yield Stable LNPs

A design of experiment (DoE) was used to model the effect of the cationic calixarene CX12 mass per LNP (expressed as milligrams of CX12 per milligram of RNA dose) on physicochemical properties. The other lipids (DLin-DMA, DOPE, cholesterol, DMG-PEG) and mRNA (Firefly luciferase) masses were maintained constant. Results of the experiment and of the modeling are shown in Table 8 and FIG. 9. LNPs made with between 0.1 and 20.7 times more CX12 than mRNA were all found to be monodisperse and encapsulate more than 95% of the RNA. The addition of CX12 was found to increase both size (from 77 to 129 nm) and charge (from 5.8 to 11.0 mV).

TABLE 8
Design of experiment used to model the effect of CX12 mass per
LNP on physicochemical properties. Results of the modeling.

| CX12 | | | | | |
|---|---|---|---|---|---|
| mass per | Z-average | | RNA conc. | EE | ζ-potential |
| mg RNA | (nm) | PDI | (ng/μL) | (%) | (mV) |
| 1.7 | 83 | 0.09 | 52 | 97.6 | 4.82 |
| 0 | 87 | 0.11 | 73 | 97.2 | 3.41 |
| 20.7 | 245 | 0.18 | 73 | 98.9 | 17.53 |
| 0 | 86 | 0.08 | 65 | 96.8 | 2.33 |
| 20.7 | 79 | 0.15 | 60 | 98.2 | 4.51 |
| 20.7 | 78 | 0.09 | 57 | 98.1 | 7.18 |
| 1.7 | 85 | 0.17 | 58 | 97.9 | 7.04 |
| 6.2 | 76 | 0.1 | 69 | 99.5 | 11.04 |
| 1.7 | 72 | 0.05 | 59 | 97.9 | 7.02 |
| 0 | 93 | 0.21 | 66 | 96.8 | 2.46 |
| 6.2 | 78 | 0.12 | 66 | 99.5 | 11.5 |
| 6.2 | 77 | 0.1 | 80 | 99.6 | 11.98 |

### Example 8: Cationizable Calix[4] Arenes for Use in an Immunogenic Composition

The present discovery is not limited to cationic calix[4] arenes and the same adjuvanticity was observed for cationizable calix[4] arenes, i.e., calix[4] arenes bearing secondary or ternary amines (preferably ternary amines). CX5 was synthetized (FIG. 16), and self-assembled into hybrid nanoparticles with a helper lipid (phospholipid), a sterol and a PEGylated lipid. Unlike cationic calixarene that were self-assembled alongside an i-lipid, the cationizable calixarene was used instead of the i-lipid in these nanoparticles. The molar ratios were again optimized to produce monodisperse nanoparticles encapsulating mRNA. For the best performing nanoparticles produced with CX5 and tested as part of the optimization study, the protein expression reached only 5% of an LNP made with SM-102 or ALC-0315 as the i-lipid (1 μg of Fluc mRNA, IM administration in BALB/c mice, estimated radiance at 6 hours post-administration-data not shown).

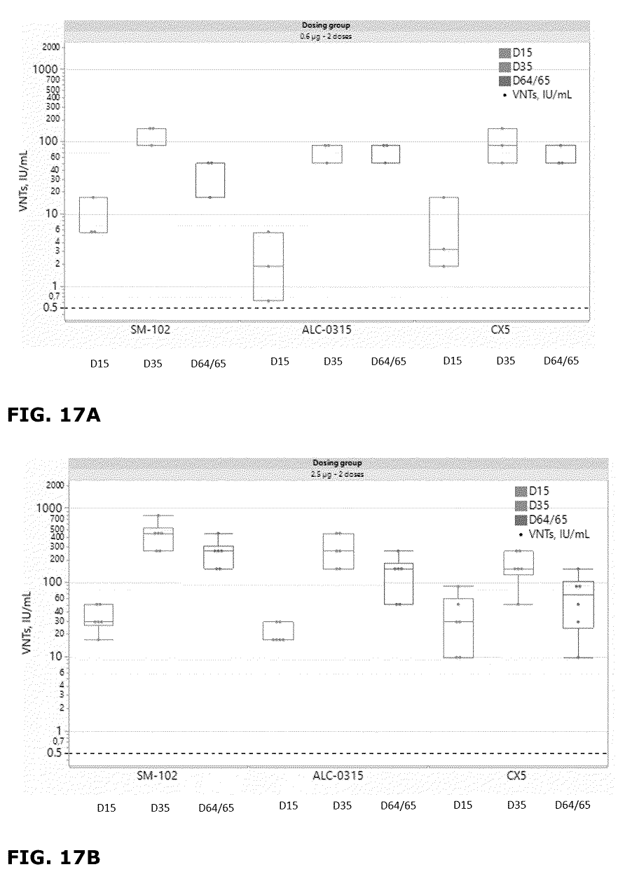

CX5 was tested in an immunogenicity study with mRNA encoding for the glycoprotein G from Rabies as a model antigen. BALB/c Mice were vaccinated following a 21 days prime-boost regimen using 0.6 μg or 2.5 μg of RNA per dose. As shown in FIG. 17,

CX5 yielded strong Virus neutralizing titers (VNTs), well above the correlate of protection (0.5 IU/mL), using the two dosing regimens (0.6 or 2.5 μg). After 15 days (after priming), the response induced by CX5 was comparable to the one obtained with two comparators (SM-102 LNPs and ALC-0315 LNPs), whatever the dosing regimen (0.6 or 2.5 μg). At 0.6 μg, this response remained comparable to the SM-102 and ALC-0315 LNPs after the boost (35 days) and at the end study (65 days). On the contrary, at 2.5 μg, the two comparators were shown to slightly outperform CX5 after the boost (35 and 65 days). The observation that protein expression is 20 times lower for CX5 as compared to other LNPs, but still reaches similar or only slightly lower VNTs, is another demonstration that calix[4] arenes, in this case cationizable calix[4] arenes can be used in potent immunogenic compositions having an adjuvant effect for nucleic acid-based vaccines.

It is worth mentioning that the nanoparticle made with ionizable calixarenes were shown to induce no adverse effect (loss of weight, impact on spleen, liver and kidneys weight, inflammation or an excessive reactogenic response) on the animals that were used during the in vivo studies.

The present invention is in no way limited to the embodiments described in the examples and/or shown in the figures. On the contrary, methods according to the present invention may be realized in many different ways without departing from the scope of the invention.

1. A delivery system to deliver one or more cargo to one or more cells, wherein the delivery system comprises a calixarene, a phospholipid, and an additional lipid wherein the additional lipid is selected from a sterol, a fatty acid, a glycerol monooleate, a trioleate or a short saturated molecule.

2. The delivery system according to claim 1, wherein said calixarene is a calix[4] arene.

3. The delivery system according to claim 1, wherein said calixarene is an ionizable calixarene, a cationic calixarene or any combination thereof.

4. The delivery system according to claim 1, further comprising an ionizable lipid.

5. The delivery system according to claim 1, further comprising a PEGylated lipid and/or a polysarcosine.

6. The delivery system according to claim 1, wherein said system comprises a calixarene, a phospholipid, a sterol and a PEGylated lipid, wherein said calixarene is an ionizable calixarene.

7. The delivery system according to claim 6, wherein said calixarene is a calix[4] arene bearing one or more ionizable head groups.

8. The delivery system according to claim 7, wherein one or more of said ionizable head groups comprises ternary amine groups.

9. The delivery system according to claim 7, wherein said calixarene comprises 4 identical ionizable head groups.

10. The delivery system according to claim 6, wherein said ionizable calixarene is present in said delivery system at a mass fraction of 10-60% (w/w).

11. The delivery system according to claim 6, wherein said phospholipid is present in said delivery system at a mass fraction of 5-35% (w/w).

12. The delivery system according to claim 6, wherein said sterol is present in said delivery system at a mass fraction of 15-50% (w/w).

13. The delivery system according to claim 6, wherein said PEGylated lipid is present in said delivery system at a mass fraction of 2-24% (w/w).

14. The delivery system according to claim 1, wherein said system comprises a calixarene, wherein said calixarene is a cationic calixarene; a phospholipid; a sterol; a PEGylated lipid; and an ionizable lipid.

15. The delivery system according to claim 14, wherein said calixarene is a calix[4] arene bearing 4 cationic head groups.

16. The delivery system according to claim 15, wherein one or more of said cationic head groups comprises at least one quaternary amine group.

17. The delivery system according to claim 15, wherein said calixarene comprises 4 identical cationic head groups.

18. The delivery system according to claim 14, wherein said cationic calixarene is present in said delivery system at a mass fraction of 0.1-50% (w/w).

19. The delivery system according to claim 14, wherein said phospholipid is present in said delivery system at a mass fraction of 5-35% (w/w).

20. The delivery system according to claim 14, wherein said sterol is present in said delivery system at a mass fraction of 15-50% (w/w).

21-80. (canceled)
